(19) United States
(12) Patent Application Publication
(10) Pub. No.: US 2015/0321168 A1
(43) Pub. Date: Nov. 12, 2015

(54) CARBON NANOTUBE PONYTAILS

(71) Applicant: UNIVERSITY OF NOTRE DAME DU LAC, Notre Dame, IN (US)

(72) Inventors: Chongzheng Na, South Bend, IN (US); Haitao Wang, South Bend, IN (US); Hanyu Ma, South Bend, IN (US)

(73) Assignee: UNIVERSITY OF NOTRE DAME DU LAC, Notre Dame, IN (US)

(21) Appl. No.: 14/709,173

(22) Filed: May 11, 2015

Related U.S. Application Data
(60) Provisional application No. 61/990,785, filed on May 9, 2014.

(51) Int. Cl. B01J 20/20; B01J 20/08; B01J 20/28; B01J 21/18; B01J 23/02; B01J 35/02; B01J 35/06; B01J 37/03; B01J 37/02; C09K 3/32; A61L 2/23; C02F 1/28; B01D 15/26; B01D 61/00; C02F 1/70; C02F 1/48; B01D 69/02; C02F 1/44; B01J 20/22

(52) U.S. Cl. CPC B01J 20/205; B01J 20/22; B01J 20/08; B01J 20/28023; B01J 20/28061; B01J 20/28064; B01J 21/185; B01J 23/02; B01J 35/023; B01J 35/06; B01J 37/031; B01J 37/0215; C09K 3/32; A61L 2/23; C02F 1/283; B01D 15/265; B01D 61/00; C02F 1/70; C02F 1/48; B01D 69/02; C02F 1/44; C02F 2101/308

(57) ABSTRACT

Carbon nanotubes (CNTs) are promising nanomaterials that have the potential to revolutionize water and waste treatment practices in the future. The direct use of unbounded CNTs, however, poses health risks to humans and ecosystems because they are difficult to separate from treated water. Here, we report the design and synthesis of carbon nanotube ponytails (CNPs) by integrating CNTs into micrometer-sized particles, which greatly improves the effectiveness of post-treatment separation using gravitational sedimentation, magnetic attraction, and membrane filtration. We further demonstrate that CNPs can effectively perform major treatment tasks, including adsorption, disinfection, and catalysis. Using model contaminants, such as methylene blue, Escherichia coli, and p-nitrophenol, we show that all the surfaces of individual CNTs in CNPs are accessible during water treatment. Hierarchical structures containing CNPs can be employed in a multitude of nano-material engineering applications, such as water and waste treatment.

### RELATED APPLICATIONS

This application claims priority under 35 U.S.C. §119(e) to U.S. Provisional Patent Application No. 61/990,785, filed on May 9, 2014, which is incorporated herein by reference.

### GOVERNMENT SUPPORT

This invention was made with government support under Grant No. CBET-1033848 awarded by the National Science Foundation and Grant No. CFP-12-3923 awarded by the Department of Energy. The government has certain rights in the invention.

### BACKGROUND OF THE INVENTION

Carbon-based materials are widely used in water and gas purification as well as food processing and drug production. Rodriguez-Reinoso, F., Activated Carbon and Adsorption. In Encyclopedia of Materials: Science and Technology, 2nd ed.; Jürgen Buschow, K. H.; Robert, W. C.; Merton, C. F. The most common carbon-based material is activated carbon produced by pyrolysis of precursors, such as nutshell, coconut husk, and peat. Activated carbon often takes the form of porous colloidal particles, which consist of tortuous channels aligned with nanometer-sized graphitic nanocrystals. In 2011, the world-wide consumption of activated carbon reached about 1.2 million metric tons with sales worth about $2 billion U.S. dollars (comparable to the gross domestic product of a country like Maldives). The global market of activated carbon is predicted to grow at a compound annual rate of 10% in the next five years. Tech Archival, Global Activated Carbon Market Assessment & Future Opportunities 2008-2018. Portland, Oreg., 2013. Among all applications, the application in municipal and industrial water treatment dominates the use of activated carbon. The Freedonia Group, World Activated Carbon: Industry Study with Forecasts for 2016 & 2021. Cleveland, Ohio, 2012.

Carbon nanotubes (CNTs) have attracted increasing attention as potential substitutes for activated carbon. Many believe that applications of nanomaterials, such as CNTs, may lead to game-changing transformations of water treatment technologies in the future. Cleaning up Water. Nat. Mater. 2008, 7, 341-341; Shannon et al., Science and Technology for Water Purification in the Coming Decades. Nature 2008, 452, 301-310; Qu et al., Nanotechnology for a Safe and Sustainable Water Supply: Enabling Integrated Water Treatment and Reuse. Acc. Chem. Res. 2013, 46, 834-843; Liu et. al., Application potential of carbon nanotubes in water treatment: A review, J. Environ. Sci. 2013, 25(7), 1263-1280. Applications of nanomaterials can particularly benefit people in impoverished countries that do not currently have water treatment infrastructures. A Fresh Approach to Water. Nature 2008, 452, 253-253; United Nations World Water Assessment Programme, The United Nations World Water Development Report 4: Managing Water under Uncertainty and Risk. Paris, France, 2012; James Ayre, Plasma-treated Carbon Nanotube Filters for Water Purification in Developing Countries, Clean Technica, Aug. 26, 2013.

CNTs are made of rolls of carbon sheets that have diameters in the nanometer range but lengths varying from tens of nanometers up to a few centimeters. Iijima, S., Helical Microtubules of Graphitic Carbon. Nature 1991, 354, 56-58. Depending on the number of carbon rolls, carbon nanotubes are categorized as single-walled, few-walled, and multi-walled CNTs. CNTs can provide a wide range of functions in water treatment, including adsorbing chemical pollutants, disinfecting pathogenic microorganisms, and supporting catalysts for contaminant degradation. De Volder et al., Carbon Nanotubes: Present and Future Commercial Applications. Science 2013, 339, 535-539; Rao et al, Sorption of Divalent Metal Ions from Aqueous Solution by Carbon Nanotubes: A Review. Sep. Purif. Technol. 2007, 58, 224-231; Pan, B.; Xing, B. S., Adsorption Mechanisms of Organic Chemicals on Carbon Nanotubes. Environ. Sci. Technol. 2008, 42, 9005-9013; Wang et al., A Comparative Study on the Adsorption of Acid and Reactive Dyes on Multiwall Carbon Nanotubes in Single and Binary Dye Systems. J. Chem. Eng. Data 2012, 57, 1563-1569; Wang et al, Synergistic and Competitive Adsorption of Organic Dyes on Multiwalled Carbon Nanotubes. Chem. Eng. J. 2012, 197, 34-40; Li et al., Antimicrobial Nanomaterials for Water Disinfection and Microbial Control: Potential Applications and Implications. Water Res. 2008, 42, 4591-4602; Serp et al., Carbon Nanotubes and Nanofibers in Catalysis. Appl. Catal., A 2003, 253, 337-358.

Compared to activated carbon, whose microscopic pores are often blocked during adsorption, CNTs' open structure offers easy, undisrupted access to reactive sites located on nanotubes' outer surfaces. Although activated carbon often has higher specific surface areas (500-1000 m² g⁻¹) than CNTs (433 m² g⁻¹ or less for CNTs with more than one wall), CNTs frequently exhibit higher capacity and faster kinetics in sorption than activated carbon, which have been attributed to the rapid transfer of contaminants from water to CNT surfaces due to CNTs' open structure. Pan, B.; Xing, B., Adsorption Mechanisms of Organic Chemicals on Carbon Nanotubes. Environ. Sci. Technol. 2008, 42, 9005-9013; Yang et al., Aqueous Adsorption of Aniline, Phenol, and Their Substitutes by Multi-Walled Carbon Nanotubes. Environ. Sci. Technol. 2008, 42, 7931-7936; Hameed et al., Adsorption of Methylene Blue onto Bamboo-Based Activated Carbon: Kinetics and Equilibrium Studies. J. Hazard. Mater. 2007, 141, 819-825; Yan et al., Adsorption of Microcystins by Carbon Nanotubes. Chemosphere 2006, 62, 142-148; El-Sheikh et al., Critical Evaluation and Comparison of Enrichment Efficiency of Multi-Walled Carbon Nanotubes, C18 Silica and Activated Carbon Towards Some Pesticides from Environmental Waters. Talanta 2008, 74, 1675-1680.

Furthermore, CNTs' open structure also provides convenience for modifying surface chemistry. The sp²-hybridized C atoms in CNTs can be readily converted to sp³-hybridized C atoms, which can host surface functionalities such as hydroxyl (—OH) and carboxyl (—COOH) groups to improve adsorption selectivity.

In spite of CNTs' exciting properties, the direct use of CNTs in water treatment has not been meaningfully developed due to limitations in the technology, including the challenge of recollecting CNTs after treatment. Conventionally, powdered activated carbon (PAC) particles are collected by gravitational sedimentation, filtration, or coagulation after use. Snoeyink, V. L.; Summers, R. S., Chapter 13: Adsorption of Organic Compounds. In Water Quality and Treatment: A Handbook of Community Water Supplies, Letterman, R. L., Ed. McGraw-Hill: New York, 1999. Different from PAC, none of the conventional techniques are expected to work well for CNTs.

CNTs do not settle well under gravity due to their small sizes. With their small sizes, CNTs can cause clogging of filtration membranes and packed beds. Although coagulation can separate CNTs, mixing them with coagulants makes it difficult to recycle, regenerate, and reuse the expensive material (ca. $100 per kg for CNTs vs. $1.5 per kg for activated carbon). CNTs left in treated water not only incur costs for replenishment, but also cause concerns for potential adverse effects on human health and the health of ecosystems. Colvin, V. L., The Potential Environmental Impact of Engineered Nanomaterials. Nat. Biotechnol. 2003, 21, 1166-1170; Lam et al., A Review of Carbon Nanotube Toxicity and Assessment of Potential Occupational and Environmental Health Risks. Crit. Rev. Toxicol. 2006, 36, 189-217; Aschberger et al., Review of Carbon Nanotubes Toxicity and Exposure-Appraisal of Human Health Risk Assessment Based on Open Literature. Crit. Rev. Toxicol. 2010, 40, 759-790; Lowry et al., Environmental Occurrences, Behavior, Fate, and Ecological Effects of Nanomaterials: An Introduction to the Special Series. J. Environ. Qual. 2010, 39, 1867-1874; Lee et al., Nanomaterials in the Construction Industry: A Review of Their Applications and Environmental Health and Safety Considerations. ACS Nano 2010, 4, 3580-3590.

A potential solution to CNTs' recollection challenge is to attach CNTs on colloidal particles made of supporting materials, such as aluminum oxide and silicon carbide. He et al., Diameter- and Length-Dependent Self-Organizations of Multi-Walled Carbon Nanotubes on Spherical Alumina Microparticles. Carbon 2010, 48, 1159-1170; Kim et al., Sub-Millimeter-Long Carbon Nanotubes Repeatedly Grown on and Separated from Ceramic Beads in a Single Fluidized Bed Reactor. Carbon 2011, 49, 1972-1979; Kim et al., Fluidized-Bed Synthesis of Sub-Millimeter-Long Single Walled Carbon Nanotube Arrays. Carbon 2012, 50, 1538-1545; Li et al., The Controlled Formation of Hybrid Structures of Multi-Walled Carbon Nanotubes on Sic Plate-Like Particles and Their Synergetic Effect as a Filler in Poly(Vinylidene Fluoride) Based Composites. Carbon 2013, 51, 355-364.

Although attaching CNTs on large, heavy particles improves collectability, the final composite product only has CNTs as a minor component in terms of mass and/or volume. Transporting non-reactive supports with a large mass over distances and dispersing them in water waste energy. Placing supports with a large volume in packed beds wastes space. To develop colloidal CNT composites for water treatment, the challenge is to design a hierarchical structure that has not only an increased overall size, but also high CNT mass and volume fractions. To our knowledge, there is no report in the literature that has described any design strategy to achieve these seemingly contradictory goals.

Accordingly, there is a need for improved CNTs that can be efficiently bounded to a support and provide improved properties, including advantageous flow properties. There is also a need for improved CNTs that can be effectively separated and collected after their use. There is yet another need for improved CNTs that are multi-functional, re-collectable, and renewable. Finally, there is a need for improved CNTs that can be used to design hierarchical articles capable of developing nano-materials for a multitude of engineering applications, such as water treatment, waste treatment, oil separations, energy storage, etc. The invention disclosed herein meets these needs.

### SUMMARY

The invention provides a means to produce CNT colloidal particles that are hundreds of micrometers in size and have CNT mass and volume fractions of nearly 100%. We designate these particles as carbon nanotube ponytails (CNPs). CNPs are synthesized by growing CNT arrays of hundreds of micrometers in length on nanometer-thin mineral discs. Like individual CNTs, CNPs can be synthesized using thermal chemical vapor deposition (CVD). Different from unbounded CNTs, however, CNPs can be separated more effectively using common techniques, such as gravitational sedimentation, magnetic attraction, and membrane filtration. We further show that CNPs can perform major water treatment tasks effectively as sorbent, disinfectant, and catalyst support. Evaluations of treatment performance provided herein evince improved properties, including that the structural transformation of CNTs into CNPs does not sacrifice the accessibility of CNTs' surface, thus, maintaining important advantages of CNTs over conventional materials, such as activated carbon and clay particles.

One embodiment of the invention provides carbon nanotube ponytail (CNP) particles comprising a carbon nanotube (CNT) array integrated onto a support and having:

(a) a mass ratio of CNT array:support of greater than about 90%;

(b) a volume ratio of CNT array:support of greater than about 90%; and

(c) a CNP particle length of about 1-500 μm.

A particular embodiment of the invention provides carbon nanotube ponytail (CNP) particles comprising a carbon nanotube (CNT) array integrated onto a support and having:

(a) a mass ratio of CNT array:support of greater than about 95%, preferably, greater than about 99%;

(b) a volume ratio of CNT array:support of greater than about 95%, preferably, greater than about 99%; and

(c) a CNP particle length of about 1-500 μm, preferably, about 1-200 μm, and more preferably, about 50-200 μm.

In certain embodiments of the invention, each CNP particle comprises two arrays of CNT particles that are entangled and integrated onto the support.

In particular embodiments of the invention, the support comprises layered double oxide (LDO). The LDO support may be derived from layered double hydroxide (LDH). The LDO support may comprise cobalt or iron nanoparticles. In a particular embodiment of the invention, the LDO support comprises cobalt nanoparticles. In certain embodiments of the invention, the LDO support further comprises magnesium and aluminum particles. The magnesium and aluminum particles can be locked in the oxide lattice and decorated with cobalt or iron nanoparticles. In some embodiments of the invention, the LDO support further comprises precious and/or noble metal nanoparticles.

In particular embodiments of the invention, the CNP particles may be functionalized with oleophillic moieties.

The invention is useful for providing a water or waste treatment system comprising the above described CNP particles.

The invention also provides a method of treating a contaminated liquid comprising exposing the contaminated liquid to CNP particles and separating the treated liquid from the CNP particles, wherein the CNP particles comprise a CNT array integrated onto a support and have:

(a) a mass ratio of CNT array:support of greater than about 90%;

(b) a volume ratio of CNT array:support of greater than about 90%; and

(c) a CNP particle length of about 1-500 μm.

The separation step of the method of the invention may be accomplished by (i) gravitational sedimentation, (ii) magnetic attraction, (iii) membrane filtration, or (iv) a combination thereof. The method of treatment may involve adsorbing the contaminants onto the CNP particles or catalyzing the contaminants to a less noxious state. In certain embodiments of the method of the invention, the CNP particles are separated, collected and regenerated for re-use via a solvent wash or thermal exposure.

The invention also provides a method of making CNP particles comprising:

(i) co-precipitating aluminum, magnesium, and cobalt or iron cations with hydroxide and carbonate anions to form a LDH support;

(ii) reducing the LDH support to a LDO support; and

(iii) growing an entangled CNT array that is integrated onto the LDO support to form the CNP particles; wherein the CNP particles have:

(a) a mass ratio of CNT array:support of greater than about 90%;

(b) a volume ratio of CNT array:support of greater than about 90%; and

(c) a CNP particle length of about 1-500 μm.

The LDH discs may be prepared by mixing and heating a solution of nitrate salts of aluminum, magnesium, and cobalt or iron with urea in deionized water. The LDO discs may be prepared by dehydrating and decarbonating the LDH discs. The CNT particles may be prepared by using ethanol as a carbon source. Other sources of carbon include, but are not limited to, methanol, carbon monoxide, methane, ethylene, benzene, and the like (e.g., most volatile hydrocarbons).

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Synthesis of carbon nanotube ponytails: (1) formation of layered double hydroxide (LDH) discs, (2) transformation of LDH to layered double oxide (LDO) discs, and (3) growth of carbon nanotube arrays on LDO.

FIG. 2A-G. Carbon nanotube ponytails: (A, B) Scanning electron micrographs (layered double oxide discs marked by arrows); (C, F) Transmission electron micrographs of CNTs in CNPs; (D) Raman spectrum; (E) X-ray photoelectron spectrum; (G) Magnetic loop of CNPs. Scale bars: A, 3 μm; B, 1 μm; C, 100 nm; F, 5 nm.

FIG. 3A-C. Specific surface area of carbon nanotube ponytails (CNPs): (A) Representative adsorption (squares) and desorption (circles) of N₂ at 77K expressed in the total surface area of N₂ per gram of carbon nanotube ponytails CNPs vs. the normalized N₂ pressure. The solid curve is a least-square fit to the BET equation (see text for details); (B) Pore size distribution calculated by the Non-Local Density Functional Theory; (C) Correlation of the specific surface area obtained by fitting N₂ adsorption to the BET equation with that computed from the morphological dimensions of CNPs (see text for details). The solid line is a least-square linear regression (S_{BET}=1.01(±0.03)S_{cal}, R²=0.99). The dashed lines are the confidence intervals corresponding to one standard deviation. The circle marks the sample used for further evaluation of separation and water treatment.

FIG. 4A-C. Separation of CNPs (bottom line), compared to unbounded CNTs (top line), from clean water by: (A) gravitational sedimentation (circles); (B) magnetic attraction (squares); and (C) membrane filtration (diamonds). The curves are least-square regressions of different separation models (see text for details).

FIG. 5A-F. Adsorption of methylene blue by carbon nanotube ponytails: (A) Kinetics of methylene blue (MB) adsorption. Symbols: triangles, Cₒ=30 mg L⁻¹; squares, Cₒ=60 mg L⁻¹; diamonds, Cₒ=200 mg L⁻¹; (B) Adsorption isotherm of methylene blue measured after 4-hr incubation. The solid lines are linear regressions. The dashed lines are 95% confidence intervals. Symbols: cyan, pH 4; crimson, pH 6; green, pH 8; purple, pH 10; (C) Desorption of 10 mg CNPs using 3 consecutive cycles of 15 mL ethanol wash. Symbols: circles, 1st cycle; squares, 2nd cycle; triangles, 3rd cycle; (D) Recovery of CNPs' occupied sites in five adsorption-desorption cycles; (E) Recovery of CNPs' occupied sites using microwave heating. Symbols: circles, 1st cycle; squares, 2nd cycle; triangles, 3rd cycle; (F) Transmission electron micrograph of microwave-irradiated used CNPs. Arrows: graphitic sheets formed by adsorbed MB. Scale bar: 50 nm.

FIG. 6A-D. Removal of E. coli by carbon nanotube ponytails: (A) Decrease of the number of survived E. coli. in the log unit with increasing CNP dosage X; (B) Adsorption isotherm of E. coli. The solid line is a least-square linear regression. The dashed lines are 95% confidence intervals; (C, D) Scanning electron micrographs of CNPs after the adsorption of E. coli. Arrows: 1, dehydrated loose cell; 2, wrapped whole cell; 3, wrapped cell fragment. Scale bars: 2 μm.

FIG. 7A-E. Catalytic reduction of p-nitrophenol (PNP) by carbon nanotube ponytails and enhancement of catalytic performance with decoration of palladium (Pd) nanoparticles; Pd-CNPs and PNP with excess sodium borohydride (SB; lowest line, angling downward), CNPs and PNP with excess SB (upper line, horizontal) and PNP and SB only (middle line, slightly angling downward): (A) Decrease of PNP concentration C with respect to the initial concentration C₀ with time; (B, C) Transmission electron micrographs of Pd-decorated carbon nanotubes removed from Pd-CNPs by sonication; (D) Fast Fourier transform of b; (E) A molecular model matching b. Scale bars: b, 20 nm; c, 1 nm; d, 4 nm⁻¹.

FIG. 8A-B. Powder X-ray diffraction patterns of layered double hydroxide and layered double oxide discs. Powder X-ray diffraction patterns of (A) layered double hydroxide (LDH) and (B) layered double oxide (LDO) discs. The patterns confirm that LDH has a hydrotalcite structure and LDO has a spinel structure.

FIG. 9A-I. Control of physical dimensions of carbon nanotube ponytails (CNPs) by varying synthesis time and cobalt doping. Control of physical dimensions of carbon nanotube ponytails (CNPs) by varying synthesis time and cobalt doping (α=[Co]/([Co]+[Mg]+[Al])). (A) Diagram showing the physical parameters including radius of CNP cross section r, CNP half-length l, carbon nanotube (CNT) outer diameter d, and CNT wall number n. (B) Example histogram for estimating r with a Gaussian fit. (C) Increase of r with reaction time t (α=13%). The solid curve represents the regression of r and t to Equation 12 using data at t≧10 hr (solid circles). The dashed curves mark the confidence interval corresponding to one standard deviation (68.3%). The horizontal solid bar at r=0 and extending from t=0 to 2 hr represents our observation of little LDH discs nor nuclei at the early stage of synthesis. (D) Invariance of r with increasing α. (E) Growth of CNPs with increasing synthesis time for chemical vapor deposition, expressed as the percentage of CNTs in CNPs. (F, G, H) Changes of l, d, and n with increasing α fitted with polynomials. (I) Nominal diameter of Co nanoparticles on LDO with fit to Equation 13.

FIG. 10A-B. (A) Transmission electron microscopy and (B) X-ray photoelectron spectrum of unbound carbon nanotubes. Scale bar: 10 nm.

FIG. 11A-B. (A) Visible spectra and (B) the absorbance-concentration relationships for carbon nanotubes (CNTs) and carbon nanotube ponytails (CNPs) dispersed in water. Concentrations of CNTs and CNPs in a are 40 and 30 mg/L, respectively. The solid lines are least-square regressions of experimental data to Equation 14. The dashed lines bracket one standard deviation of prediction.

FIG. 12A-B. (A) UV/vis spectra of p-nitrophenol (upper line, which includes the highest peaks) and p-aminophenol (lowest line, which includes the lowest peak) and (B) the absorbance-concentration relationship used for p-nitrophenol quantification.

### DETAILED DESCRIPTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages and diameter sizes) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values are for illustration only and do not exclude other defined values or other values within defined ranges.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, and the like.

The term “integrated” refers to the CNT arrays being joined or incorporated into the support. The CNT arrays may be anchored or bound to the support. CNT arrays are held strongly to the support through cobalt nanoparticles, which are fused to CNTs on one end and embedded into the support lattice on the other end.

The term “growing” is used to characterize CNT synthesis. During synthesis, new carbon atoms are added to the interface of Co-CNT, so that the length of a CNT is extended.

The term “colloidal” refers to the size of the CNP particles, which range from about 1-1,000 μm.

We have created multifunctional and re-collectable nano-sized CNPs that are useful for treating water and waste. The CNPs comprise CNTs bounded onto a support and having high weight ratios and high volume ratios. In contrast, CNTs bound to colloidal particles have low weight ratios (<50%) and/or low volume ratios (<50%). Compared to unbound CNTs of micrometers in length but nanometers in diameter, CNPs are colloidal in nature because both dimensions are in the micrometer range. Although CNTs in CNPs are located within close proximity to one another, their surfaces are completely accessible for removing contaminants in water.

In certain embodiments of the invention, the CNP particles are about 1-500 μm in length, preferably, about 1-200 μm in length, and more preferably, about 50-200 μm in length. The CNP particles are about 0.1-10 μm in diameter, preferably, about 1-5 μm in diameter, and more preferably, about 2-4 μm in diameter. The support is about 10-100 nm in thickness, preferably, about 20-80 nm in thickness, and more preferably, about 40-60 nm in thickness. The support is about 0.1-5 μm in diameter, preferably, about 1-5 μm in diameter, and more preferably, about 2-5 μm in diameter. The CNPs are tubular in shape. Both sides refer to the two sides of a pseudo 2-D LDO support. CNPs are tubular after CNT arrays are added to the support.

The outer and inner diameters are used to characterize carbon nanotubes (CNTs). The outer diameter is about 4-9 nm in size, while the inner diameter is about 3-6 nm. The walls of the CNTs may be single, few, or multi. These terms are used to describe the number of walls possessed by CNTs. In CNPs, the CNTs have about 2-10 walls, so they are few- and multi-walled CNTs.

The presence of a range of pore sizes is good for adsorbing contaminants (large pores serving as flow/diffusion paths and small ones to trap contaminant molecules). The pores of CNP particles are about 1-500 nm in diameter, preferably, about 2-250 nm in diameter, and more preferably, about 2-100 nm in diameter. In one embodiment, the CNP particles are about 4.5-100 nm in diameter. CNP particles have a SSA of about 100-750 m² g⁻¹, preferably, about 150-600 m² g⁻¹, and more preferably, about 200-600 m² g⁻¹. In one embodiment, the CNP particles have a SSA of about 200-500 m² g⁻¹. The density of CNPs is approximately that of CNTs (i.e., the mass and volume of the support is negligible), which is a function of CNT diameter and wall number. The examples described herein have a density of about 1.9(±0.5) g/cm³.

The support is a thin hexagonal disk made of a polycrystalline spinel (MgAl₂O₄). Its surfaces are decorated with Co₃O₄ nanoparticles, which during CNT synthesis are reduced to metallic Co nanoparticles to catalyze CNT growth. While the examples described herein create LDOs from Co-containing LDHs, it is also contemplated that LDOs can be made from Fe-containing LDHs. The LDH supports are similar in size and weight to the LDO supports.

The Co₃O₄ nanoparticles make the CNP particles magnetic. The 3^{rd} step of the process to make the support reduces Co₃O₄ to Co. Co is readily oxidized by oxygen in air to Co₃O₄, which occurs when CNPs are used under ambient conditions. The metal particles are nano in size catalyzing CNT growth.

The CNPs' advantageous properties make it a suitable nano-material for many engineering applications, such as water and waste treatment. Uses include treating contaminated (polluted) liquids, emulsions, suspensions, and the like. Chemical and biological pathogens, microorganisms, and the like can be treated, purified or transformed into a less toxic state (via catalysis). CNP technology can be adapted to fabricate portable and large-sized membranes, filters, cartridges, and partial or complete treatment systems. Purification and disinfection of water and treatment of fuel or waste are just some of the examples of where this technology would be beneficial. Other applications can be envisioned, such as energy storage (e.g., micro particles that store charge in flow batteries and capacitors) and catalyst engineering (e.g., CNP supports). Regarding H₂ storage, adsorption and desorption of H₂ would be expected to improve by the organization of CNTs in CNPs to create pore structures. Regarding catalyst support, the large specific surface areas should make CNPs useful because while activated carbon has greater specific surface areas, most of the surfaces are not accessible to catalyst nanoparticles. CNPs possess advantageous flow properties that make them useful for many applications.

### Synthesis and Characterization of Carbon Nanotube Ponytails

Syntheses of CNTs are described in the literature. Li et al., Synthesis of Carbon Nanotubes Using a Novel Catalyst Derived from Hydrotalcite-Like Co—Al Layered Double Hydroxide Precursor. Catal. Lett. 2005, 99, 151-156; Zhao et al., Catalytic Synthesis of Carbon Nanostructures Using Layered Double Hydroxides as Catalyst Precursors. Carbon 2007, 45, 2159-2163; Zhao et al., Embedded High Density Metal Nanoparticles with Extraordinary Thermal Stability Derived from Guest-Host Mediated Layered Double Hydroxides. J. Am. Chem. Soc. 2010, 132, 14739-14741.

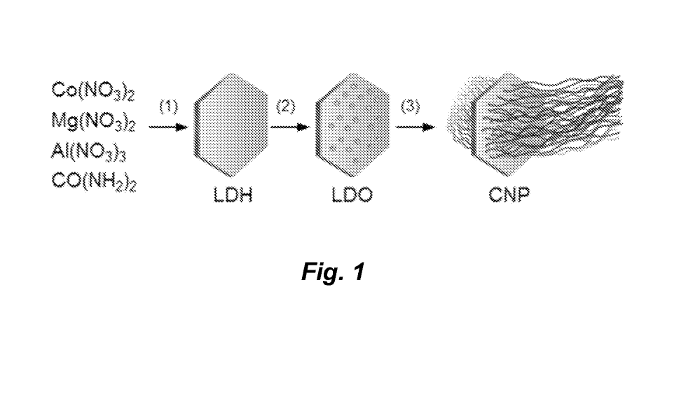

We synthesized CNPs using a three-step procedure as outlined in FIG. 1.

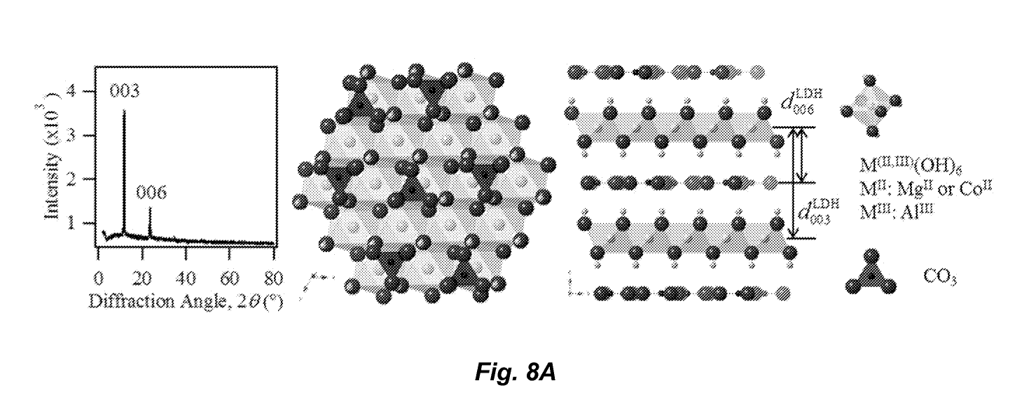

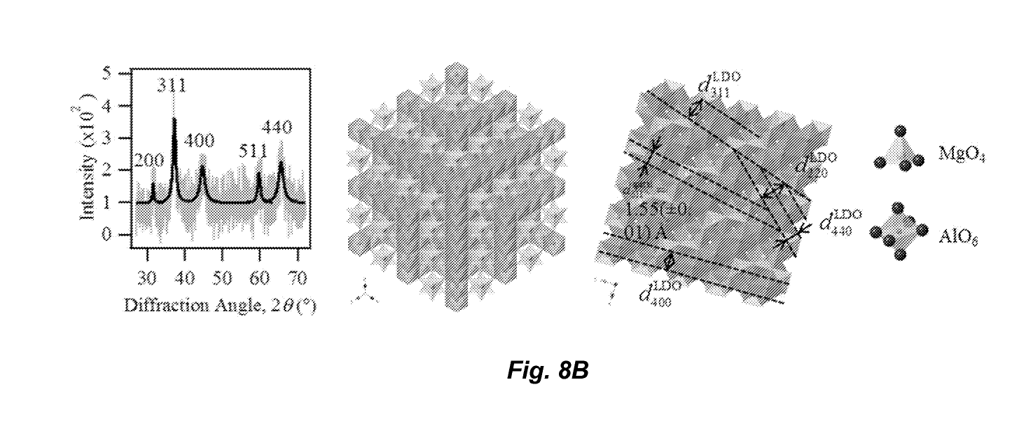

First, layered double hydroxide (LDH; FIG. 8A) discs of a few micrometers in size and approximately 50 nm in thickness were prepared by co-precipitating aluminum, magnesium, and cobalt cations with hydroxide and carbonate anions (produced by the decomposition of urea). Zhao et al., Controllable Bulk Growth of Few-Layer Graphene/Single-Walled Carbon Nanotube Hybrids Containing Fe@C Nanoparticles in a Fluidized Bed Reactor. Carbon 2014, 67, 554-563.

Second, LDH discs were transformed to layered double oxide (LDO; FIG. 8B) by dehydration and decarbonation at 800° C. in argon. The treatment produced cobalt oxide (CoO) nanoparticles through phase separation.

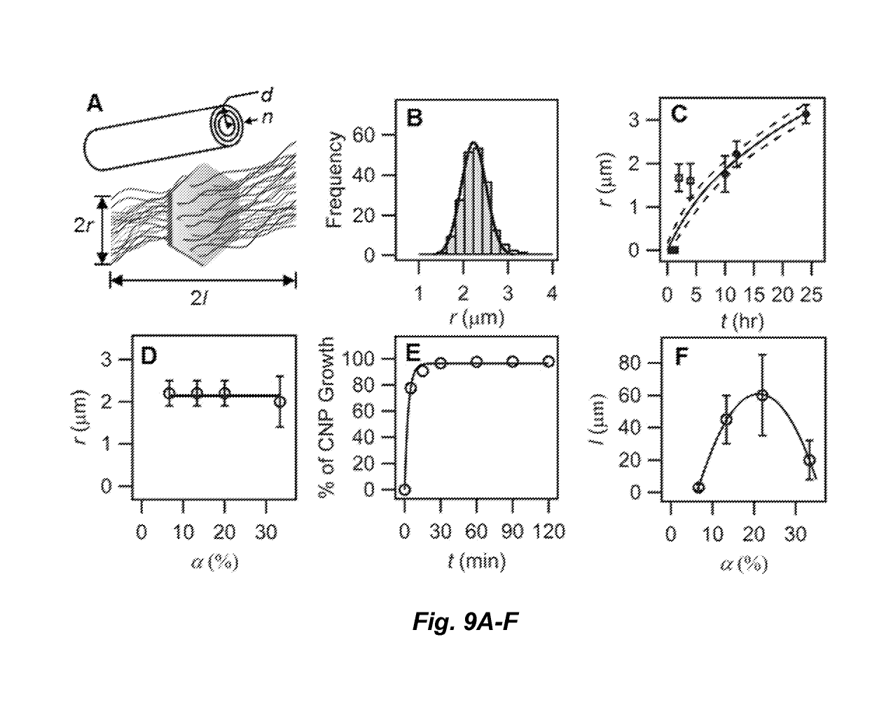

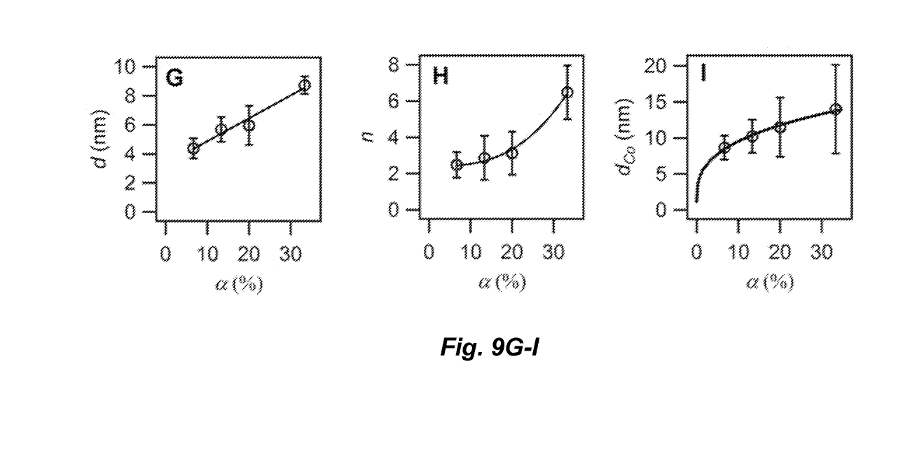

Third, CoO was reduced to Co by H₂, and then, entangled CNT arrays were grown using chemical vapor deposition (CVD) on both sides of the LDO discs at 800° C. using ethanol as the carbon source. The CNPs comprised the entangled CNT arrays grown on the support (disc). This procedure typically yielded about 70 grams of CNTs for each gram of Co catalyst (cf. FIG. 9A-I).

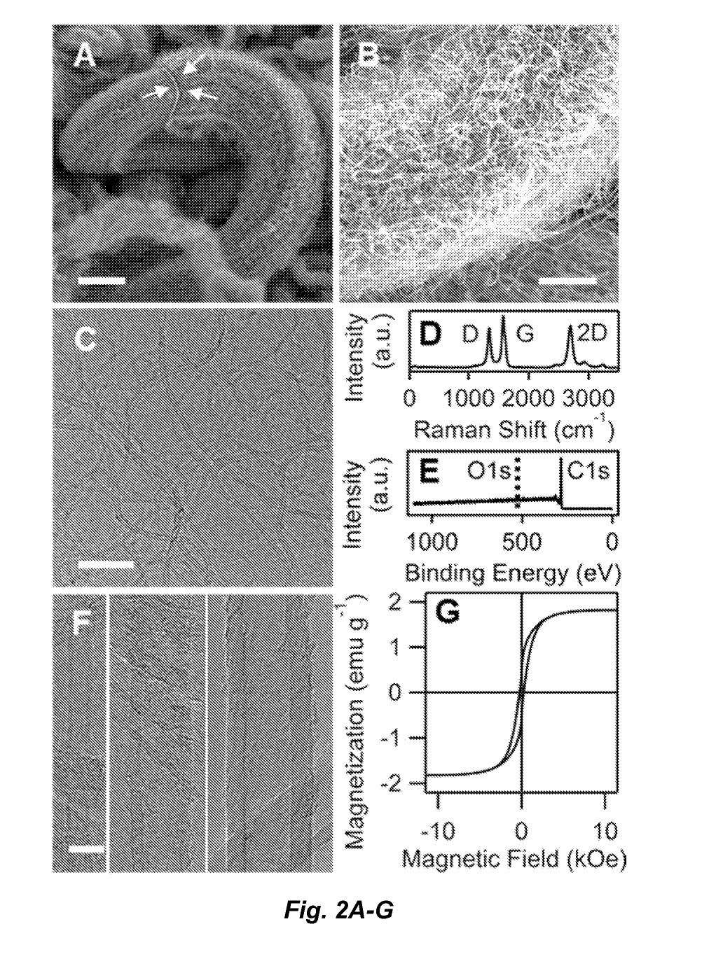

The physical properties of a typical CNP sample are shown in FIG. 2A-G. As revealed by scanning electron microscopy (SEM), a dry CNP particle has a flexible cylindrical structure with a diameter of a few micrometers and a length of tens of micrometers (FIG. 2A). Each CNP particle consists of two arrays of entangled CNTs anchored on a thin LDO disc (marked by arrows), which has a negligible contribution to the overall mass and volume. A close view shows that the CNT arrays are porous and consist of curvy nanotubes (FIG. 2B). Transmission electron microscopy (TEM) shows that individual CNTs have a relatively narrow distribution of diameters (FIG. 2C). Raman spectroscopy shows that CNTs contain defects giving a D-G ratio of 0.8 (FIG. 2D). Using the empirical relationship Lₐ=8.28/(I_{D}/I_{G}), we estimate the size of in-plane graphene crystallites at Lₐ=10.4 nm, suggesting the presence of one defect site every 10.4 nm on average. Vix-Guterl et al., Surface Characterizations of Carbon Multiwall Nanotubes: Comparison between Surface Active Sites and Raman Spectroscopy. J. Phys. Chem. B 2004, 108, 19361-19367; Delhaes et al., A Comparison between Raman Spectroscopy and Surface Characterizations of Multiwall Carbon Nanotubes. Carbon 2006, 44, 3005-3013.

Although oxidation can form defects on CNT surfaces, see e.g., Jiang et al., The Preparation of Stable Metal Nanoparticles on Carbon Nanotubes Whose Surfaces Were Modified During Production. Carbon 2007, 45, 655-661, the defects seen here were likely not formed by oxidation because little oxygen was found by X-ray photoelectron spectroscopy (FIG. 2E). The lack of surface oxygen indicates that CNPs are hydrophobic. High-resolution TEM further reveals that the outer diameter and wall number of individual CNTs, which can be controlled during synthesis, varied from 4 to 9 nm and from 2 to 10, respectively (FIG. 2F). Another important property of CNPs is that they are magnetic with a saturation magnetization of 1.8 emu g⁻¹ because of the presence of cobalt oxide nanoparticles in LDO (FIG. 2G). CNPs' saturation magnetization is 50 times smaller than the value for magnetite. Wang et al., Removal of Oil Droplets from Contaminated Water Using Magnetic Carbon Nanotubes. Water Res. 2013, 47, 4198-4205. The saturation magnetization is sufficiently weak to prevent CNPs from aggregating under self-attraction, but strong enough to be utilized for separation (see below).

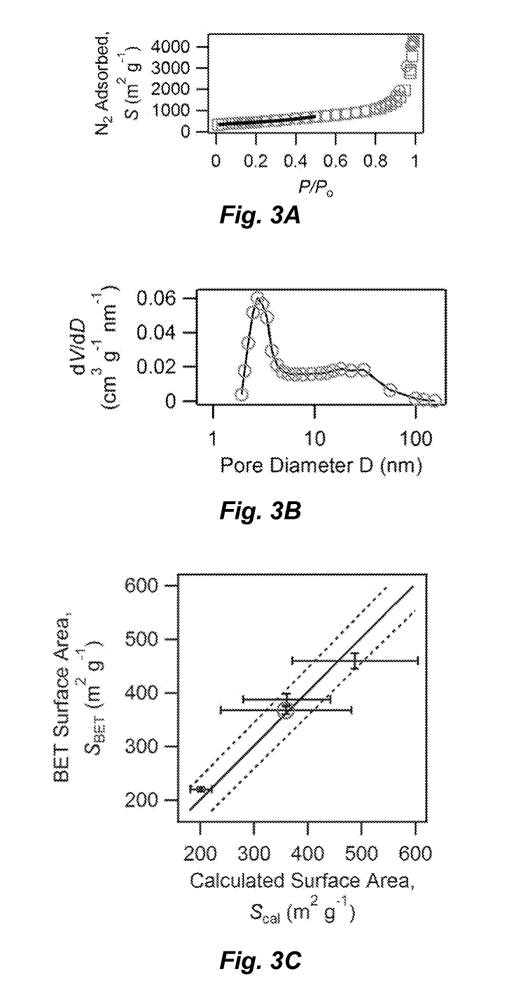

The synthesis procedure described herein allows for the control of CNPs' morphology, such as LDH size, CNT length, CNT diameter, and CNT wall number, by varying synthesis conditions (FIG. 9A-I and the accompanying text). The changes of these parameters lead to the variation of the specific surface area (SSA) of CNPs, which can be characterized by nitrogen physiosorption. As shown in FIG. 3A, a typical sorption isotherm revealed that the amount of adsorbed N₂ by each gram of CNPs, S, increased slowly at low N₂ pressures for P/Pₒ<0.6, suggesting a weak N₂-CNT interaction. As P/Pₒ becomes greater than 0.8, S increases rapidly with increasing P/Pₒ, suggesting an improved adsorption due to a strong N₂—N₂ interaction. Moreover, the lack of hysteresis between the desorption and adsorption isotherms indicates little resistance for mass transfer. These characteristics are consistent with a Type III behavior for a highly porous material. Adamson, A. W., Physical Chemistry of Surfaces. John Wiley & Sons: New York, 1990. Indeed, the pore size distribution calculated by the Non-Local Density Functional Theory reveals a broad range of pores with diameters spanning from 2 to 100 nm, as shown in FIG. 3B. We assigned the peak at 2.9(±1.3) nm to the adsorption of N₂ by the N₂-CNT interaction around individual CNTs, which is consistent with the CNT diameter of 4-7 nm calculated from nanotube dimensions. We assigned the broad band between 4.5 and 100 nm to the adsorption of N₂ by the N₂—N₂ interaction and accommodated by CNPs' porous structure.

We estimate the SSA of CNPs, S_{BET}, using the Brunauer-Emmett-Teller (BET) equation: [S(Pₒ/P−1)]⁻¹=(1−1/c)S_{BET}⁻¹(P/Pₒ)+S_{BET}⁻¹c⁻¹, where c is the BET constant. Brunauer, S.; Emmett, P. H.; Teller, E., Adsorption of Gases in Multimolecular Layers. J. Am. Chem. Soc. 1938, 60, 309-319. Using the monolayer portions of the adsorption and desorption curves (P/Pₒ<0.5), we obtain 365(±10) m² g⁻¹ through least-square regression. Similarly, the SSAs are obtained for three other CNP samples prepared under different synthesis conditions. The values of S_{BET} range from 200 to 500 m² g⁻¹, comparable to the typical surface areas of activated carbon. Snoeyink, V. L.; Summers, R. S., Chapter 13: Adsorption of Organic Compounds. In Water Quality and Treatment: A Handbook of Community Water Supplies, Letterman, R. L., Ed. McGraw-Hill: New York, 1999. As shown in FIG. 3C, these values are compared to the SSAs computed from the physical dimensions of CNTs in each sample: S_{cal}=4d⁻¹ρ⁻¹, where d is the CNT diameter and ρ is the CNT density. Chiodarelli et al., Correlation between Number of Walls and Diameter in Multiwall Carbon Nanotubes Grown by Chemical Vapor Deposition. Carbon 2012, 50, 1748-1752. Values of S_{BET} and S_{cal} agree well with each other, as evident from the linear correlation with a slope of unity, suggesting that CNPs have an open structure when they are dry.

### Separation of Carbon Nanotube Ponytails

To evaluate CNPs' performance in separation, we selected the CNP sample with a LDH size of 2.0(±0.2) μm, a CNT length of 60(±25) μm, a CNT diameter of 6.0(±1.4) nm, a CNT wall number of 4(±1), and S_{BET}=365(±10) m² g⁻¹ (marked by the circle in FIG. 3C). This sample has a CNT density of

$$ρ = 3.04 [ n \text{/} d - ( 0.34 {\sum }_{i = 0}^{n - 1} i ) \text{/} {d}^{2} ] = 1.9 ( \pm 0.5 ) g {cm}^{- 3} .$$

Laurent et al., The Weight and Density of Carbon Nanotubes Versus the Number of Walls and Diameter. Carbon 2010, 48, 2994-2996. Quantitative assessments of CNPs' behavior in the common separation processes, including (a) gravitational sedimentation, (b) magnetic separation, and (c) membrane filtration, were undertaken.

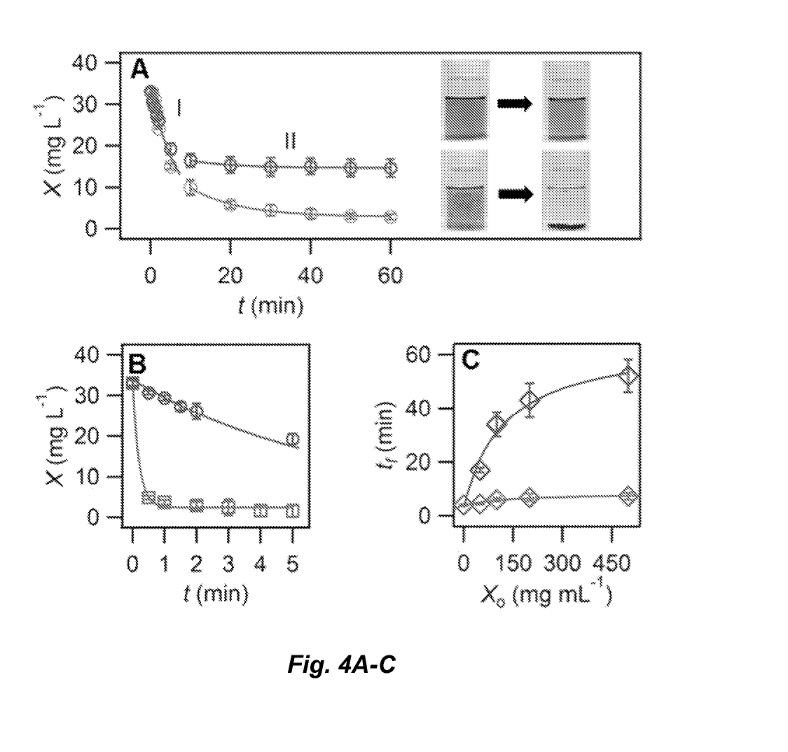

Gravitational sedimentation is widely used in both large-scale facilities and personal devices for water purification. Gregory et al., Chapter 11: Sedimentation and Flotation. In Water Quality and Treatment: A Handbook of Community Water Supplies, Letterman, R. L., Ed. McGraw-Hill: New York, 1999. As shown in FIG. 4A, CNPs (bottom line) continuously settle from an aqueous suspension with an initial concentration Xₒ=35 mg L⁻¹, which is comparable to the use of activated carbon in water treatment. Snoeyink, V. L.; Summers, R. S., Chapter 13: Adsorption of Organic Compounds. In Water Quality and Treatment: A Handbook of Community Water Supplies, Letterman, R. L., Ed. McGraw-Hill: New York, 1999. After 60 min, the originally opaque CNP suspension became clear. In comparison, unbounded CNTs (upper line and upper inset) with similar surface hydrophobicity did not settle well within the same period of time, as evident from the CNT suspension's opacity.

Two settling regimes were revealed by quantitative analyses of changes of carbon concentration X with time t. In Regime I, where X>15 mg L⁻¹, CNPs and CNTs behaved similarly because both of them settled as aggregates. Farley, K. J.; Morel, F. M. M., Role of Coagulation in the Kinetics of Sedimentation. Environ. Sci. Technol. 1986, 20, 187-195. In Regime II, where aggregates reduced to individual particles as X decreases, CNPs settled faster than CNTs because CNPs are bigger. The settling processes in both regimes conformed to the sedimentation model: X=Xₒe^{-(v/h)t}, where v is the settling velocity and h=1.2 (±0.1) cm is the height of suspension. Lick, W., Sediment and Contaminant Transport in Surface Waters. CRC Press: Boca Raton, 2008. Least-square regressions gave v_{I}=10.6(±0.6) cm h⁻¹ for both CNTs and CNPs, but v_{II}(CNPs)=2.2(±0.3) cm h⁻¹ and v_{II}(CNTs)=0.14(±0.03) cm h⁻¹.

For a personal water purification device (e.g., a water bottle) with a settling height of 2 cm (bottle placed horizontally), 95% of CNPs can be settled out in 2.3 hrs. For sedimentation tanks used for industrial or municipal water treatment that have depths of meters, but residence times of merely a couple of hours, gravitational sedimentation is not practical for CNP (or CNT) separation. Gregory et al., Chapter 11: Sedimentation and Flotation. In Water Quality and Treatment: A Handbook of Community Water Supplies, Letterman, R. L., Ed. McGraw-Hill: New York, 1999.

Magnetic nanomaterials, such as CNPs, can be separated using an external magnetic field. Wang et al., Removal of Oil Droplets from Contaminated Water Using Magnetic Carbon Nanotubes. Water Res. 2013, 47, 4198-4205. Magnetic separation of CNPs can be designed to be much faster than gravitational separation by using a magnetic field that induces an attractive force much stronger than gravity.

As shown in FIG. 4B, a magnetic field with an average strength of 4 kOe can separate more than 95% CNPs within less than 5 min (squares), which is much faster than separation under gravitational sedimentation (circles). Using X=Xₒe^{-(v/D)t}, where D=2.8 cm is the diameter of the vial containing CNP suspension (magnet placed on the side), the separation velocity is estimated at vₘ=5.8(±1.3) m h⁻¹. Lick, W., Sediment and Contaminant Transport in Surface Waters. CRC Press: Boca Raton, 2008. In a typical sedimentation tank with a depth of 2 m and a residence time of 2 hr, 99.7% removal of CNPs can be accomplished under vₘ. Gregory et al., Chapter 11: Sedimentation and Flotation. In Water Quality and Treatment: A Handbook of Community Water Supplies, Letterman, R. L., Ed. McGraw-Hill: New York, 1999. In addition to the rapid separation, the use of magnetic force can also avoid the trapping of CNPs at the water-air interface by surface tension under gravity (black dots on top of the water table in the lower right inset in FIG. 4A (upper right inset is the water table for CNTs)).

Membrane filtration is another option for CNT separation that is often used in laboratory experiments. Tanaka, T., Filtration Characteristics of Carbon Nanotubes and Preparation of Buckypapers. Desalin. Water Treat. 2010, 17, 193-198. FIG. 4C shows the time required to pass 50-mL aqueous suspension of CNPs or CNTs through a 0.8-μm membrane under the pulling of vacuum. As the initial carbon concentration Xₒ increases, the filtration time t_{f} increases with decreasing flow rate (Q∝1/t_{f}) for both CNPs (lower line) and CNTs (upper line). The decrease of flow rate is attributable to the formation of a porous film of CNTs or CNPs on top of the filtration membrane. The main determinant of flow reduction is the porosity of the film. The relationship between t_{f} and X can be modeled with t_{∞}−t_{f}=α(Xₒ+Xₘ)⁻¹, where t_{∞} is the time for the porosity of carbon film to reach a steady-state value, Xₘ is the equivalent carbon concentration of the filtration membrane, and α represents the hydraulic resistance of the porous film. Carman, P. C., Fluid Flow through Granular Beds. Chem. Eng. Res. Des. 1997, 75, S32-S48. According to experimental data, α_{CNTs}:α_{CNPs}=12.5, suggesting that CNPs form more loosely packed films than CNTs, and thus, can save energy and reduce clogging in filtration.

### Carbon Nanotube Ponytails in Water Treatment

The effectiveness of CNPs as sorbent, disinfectant, and catalyst support used in water treatment processes is demonstrated in this section. The demonstration was performed using the same CNP sample that had been used for the evaluation of CNP separation.

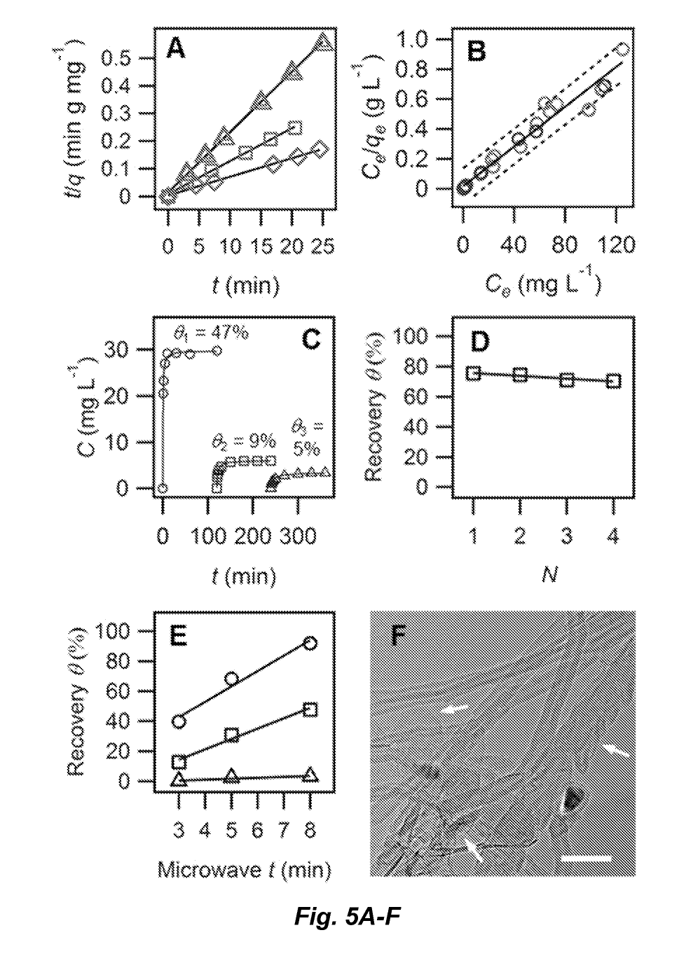

CNPs' adsorption capability was tested using methylene blue (MB) as a model pollutant. Gong et al., Removal of Cationic Dyes from Aqueous Solution Using Magnetic Multi-Wall Carbon Nanotube Nanocomposite as Adsorbent. J. Hazard. Mater. 2009, 164, 1517-1522; Yao et al., Adsorption Behavior of Methylene Blue on Carbon Nanotubes. Bioresour. Technol. 2010, 101, 3040-3046. As shown in FIGS. 5A and 5B, both kinetics and equilibrium of MB adsorption by CNPs conform to the classical Langmuir model. The kinetic study was performed at two different pH conditions and three different initial concentrations. Results can all be fitted to the linearized model: t/q=t/qₑ+1/(kₐ qₑ²), where q=(Cₒ−C)/X is the amount of MB adsorbed by CNPs at time t, Cₒ=30, 60, or 200 mg L⁻¹ is the initial MB concentration, C is the residual MB concentration at t, X=0.67 g L⁻¹ is the dose of CNPs, qₑ is the equilibrium value of q (t→∞), and kₐ is the adsorption rate constant. Liu, Y.; Shen, L., From Langmuir Kinetics to First- and Second-Order Rate Equations for Adsorption. Langmuir 2008, 24, 11625-11630.

Adsorption is insensitive to pH because MB is always a monovalent cation in the normal pH range. Pan, B.; Xing, B. S., Adsorption Mechanisms of Organic Chemicals on Carbon Nanotubes. Environ. Sci. Technol. 2008, 42, 9005-9013; Chagovets et al., Noncovalent Interaction of Methylene Blue with Carbon Nanotubes: Theoretical and Mass Spectrometry Characterization. J. Phys. Chem. C 2012, 116, 20579-20590. As shown in FIG. 5A, least-square regressions revealed that for Cₒ<30 mg L⁻¹, adsorption approached equilibrium in less than an hour (Table 1). As shown in FIG. 5B, results obtained from adsorption experiments performed for 4 hr at different pH, Cₒ, and X values conformed to the Langmuir isotherm: Cₑ/qₑ=Cₑ/qₘₐₓ+1/(K qₘₐₓ), where Cₑ is the residual MB concentration at equilibrium and qₘₐₓ is the adsorption capacity. Hiemenz, P. C.; Rajagopalan, R., Principles of Colloid and Surface Chemistry. 3rd Ed. ed.; Marcel Dekker: New York, 1997.

Regression gives qₘₐₓ=150(±9) mg g⁻¹ (Table 2). Using qₘₐₓ, the specific surface area of CNPs was computed from S_{MB}=N_{A}τqₘₐₓ/M=367(±22) m² g⁻¹, where τ=1.30 nm² is the surface area that a MB molecule occupies, M=320 g mol⁻¹ is MB's molecular weight, and N_{A}=6.02×10²³ mol⁻¹ is Avogadro's number. Hahner et al., Orientation and Electronic Structure of Methylene Blue on Mica: A near Edge X-Ray Absorption Fine Structure Spectroscopy Study. J. Chem. Phys. 1996, 104, 7749-7757; Hang, P. T., Methylene Blue Absorption by Clay Minerals: Determination of Surface Areas and Cation Exchange Capacities (Clay-Organic Studies XVIII). Clays Clay Miner. 1970, 18, 203-212; Kahr, G.; Madsen, F. T., Determination of the Cation Exchange Capacity and the Surface Area of Bentonite, Illite and Kaolinite by Methylene Blue Adsorption. Appl. Clay Sci. 1995, 9, 327-336; He et al., Adsorption and Desorption of Methylene Blue on Porous Carbon Monoliths and Nanocrystalline Cellulose. ACS Appl. Mater. Interfaces 2013, 5, 8796-8804. This equation is valid because MB forms a monolayer on the CNT surface via π-π interaction. Pan, B.; Xing, B. S., Adsorption Mechanisms of Organic Chemicals on Carbon Nanotubes. Environ. Sci. Technol. 2008, 42, 9005-9013; Chagovets et al., Noncovalent Interaction of Methylene Blue with Carbon Nanotubes: Theoretical and Mass Spectrometry Characterization. J. Phys. Chem. C 2012, 116, 20579-20590. S_{MB} agreed well with the values of S_{cal} and S_{BET}, indicating that all the surfaces of individual CNTs in CNPs were still accessible for adsorbing pollutants in water.

To assess the possibility of removing MB from CNPs by solvent wash, a multi-cycle process using ethanol was first evaluated. Gong, J. L.; Wang, B.; Zeng, G. M.; Yang, C. P.; Niu, C. G.; Niu, Q. Y.; Zhou, W. J.; Liang, Y., Removal of Cationic Dyes from Aqueous Solution Using Magnetic Multi-Wall Carbon Nanotube Nanocomposite as Adsorbent. J. Hazard. Mater. 2009, 164, 1517-1522; Ai, L. H.; Jiang, J., Removal of Methylene Blue from Aqueous Solution with Self-Assembled Cylindrical Graphene-Carbon Nanotube Hybrid. Chem. Eng. J. 2012, 192, 156-163. As shown in FIG. 5C, used CNPs with 65% surface covered (i.e., qₑ=65% qₘₐₓ) were washed in three cycles with each using 15 mL ethanol. In each cycle, the MB concentration in ethanol, C, increases from 0 and then reaches a plateau after a period of time, suggesting that the removal has reached equilibrium and fresh ethanol is necessary at the end of each cycle. After the washed CNPs were collected from ethanol by magnetic separation, they were mixed with another 15 mL fresh ethanol and the removal process was repeated. For all the cycles, the removal kinetics was found to conform to the Langmuir model (Table 3): (t−t_{o,n})/C=(t−t_{o,n})/C_{e,n}+1/(k_{d,n}C_{e,n}²), where t is time, t_{o,n} is the starting time for the nth wash, C is the MB concentration in ethanol, C_{e,n} is the equilibrium MB concentrations, and k_{d,n} is the desorption rate constant.

The percentage of freed sites by washing was computed as: θ=C_{e,n}X/q_{o,n}, where q_{o,n} is the initial concentration of MB on CNPs. For the first wash, q_{o,1} equals to qₑ=98 mg g⁻¹ (65% qₘₐₓ, obtained from the adsorption experiment). For subsequent washes, q_{o,n-1}=q_{o,n}−C_{e,n}X. As shown in FIG. 5C, θ diminishes as n increases, indicating a typical behavior of desorption equilibrium as the mechanism of MB removal in ethanol wash. After CNPs are regenerated by 10 wash cycles, θ=75% was confirmed by re-adsorbing MB, as shown in FIG. 5D (N=1). When the CNPs were used repeatedly after being administered to the adsorption-desorption (n=10) reuse cycle, θ decreases slightly after each cycle (ca. 2% reduction; Table 4), suggesting that a small fraction of CNTs were bundled together under the attraction of MB. Accordingly, a common solvent such as ethanol was inefficient for regenerating MB-laden CNPs because of a strong MB-CNT affinity and the large quantity of ethanol needed to overcome this affinity.

An alternative approach of regeneration is thermal treatment, which is regularly performed for used activated carbon. Because CNTs are good adsorbents of microwaves, thermal treatment may be performed using microwave irradiation. Yuen, F. K.; Hameed, B. H., Recent Developments in the Preparation and Regeneration of Activated Carbons by Microwaves. Adv. Colloid Interface Sci. 2009, 149, 19-27. As shown in FIG. 5E, 92% of adsorption capacity was restored after used CNPs (65% covered with MB) were irradiated in a kitchen microwave oven for 8 min under maximum power. As the number of regeneration-and-reuse cycle increased, the restored capacity started to decrease. The decrease can be attributed to the formation of graphitic sheets by adsorbed MB, as shown in FIG. 5F (marked by arrows), which destruct the organized porous structure of CNPs. The microwave-assisted thermal treatment may be further optimized to evaporate adsorbed MB without graphitizing MB.

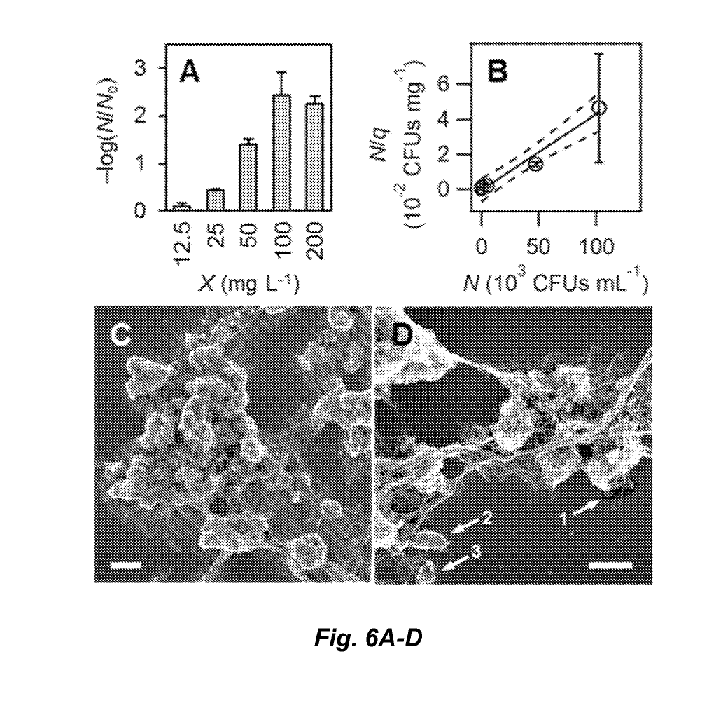

Through sorption, CNPs can be used to remove pathogenic microorganisms from water and achieve disinfection without using potentially harmful chemicals. Na, C.; Olson, T. M., Formation of Cyanogen Chloride from Glycine in Chlorination. Env. Sci. Technol. 2006, 40, 1469-1477. CNPs' potential as a disinfectant was evaluated using bacterium Escherichia coli DH5α (E. coli) as a model pathogen. The removal of E. coli from water was measured by the reduction in colony forming units (CFUs) after 1 hour of contact with CNPs. As shown in FIG. 6A, the removal of E. coli increased with the increase of CNP dosage. As shown in FIG. 6B, the removal efficiency conformed to the Langmuir model, suggesting the removal mechanism is sorption. Regression gave a sorption capacity of qₘₐₓ=2.3(±0.2)×10⁹ CFUs g⁻¹ (Table 6).

For each gram of CNPs, there were approximately 4.5(±3.7)×10⁷ CNP particles; therefore, each particle captured approximately 50 bacterial cells. If each E. coli cell is considered as a sphere with 1 μm in diameter, it is plausible for a CNP particle of 120 μm in length to catch more than 50 cells. Based on the Langmuir model, for a typical water source containing 10⁵ CFUs L⁻¹ (of which E. coli is often a minute fraction), only 46(±4) mg L⁻¹ CNPs would be required to achieve a 3-log reduction to the commonly acceptable level of 100 CFUs mL⁻¹. Hoefel et al., Enumeration of Water-Borne Bacteria Using Viability Assays and Flow Cytometry: A Comparison to Culture-Based Techniques. J. Microbiol. Methods 2003, 55, 585-597; Bartram et al., Heterotrophic Plate Counts and Drinking-Water Safety: The Significance of Hpcs for Water Quality and Human Health. IWA Publishing on behalf of the World Health Organization: London, 2003.

The capturing of bacterial cells by CNPs can be further visualized using SEM. As shown in FIG. 6C, cells were wrapped tightly by CNP particles as linked aggregates. A careful search over many SEM images revealed three types of cells, as marked in FIG. 6D, including (1) dehydrated loose cells (only one found), (2) wrapped whole cell, and (3) wrapped cell fragment. The presence of cell fragments suggests that CNPs are capable of inactivating microorganisms by damaging the cell membrane as has been seen with CNTs. Kang et al., Antibacterial Effects of Carbon Nanotubes: Size Does Matter! Langmuir 2008, 24, 6409-6413; Arias, L. R.; Yang, L., Inactivation of Bacterial Pathogens by Carbon Nanotubes in Suspensions. Langmuir 2009, 25, 3003-3012.

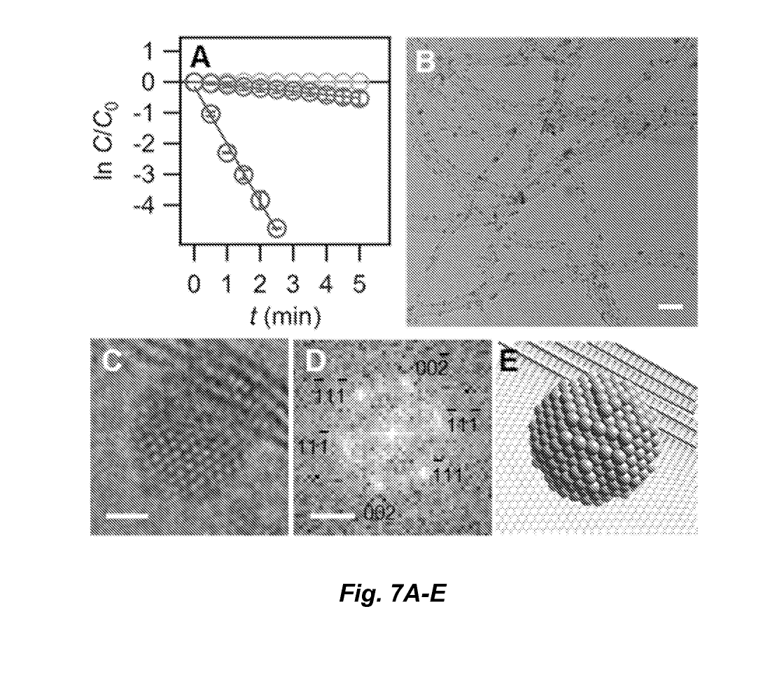

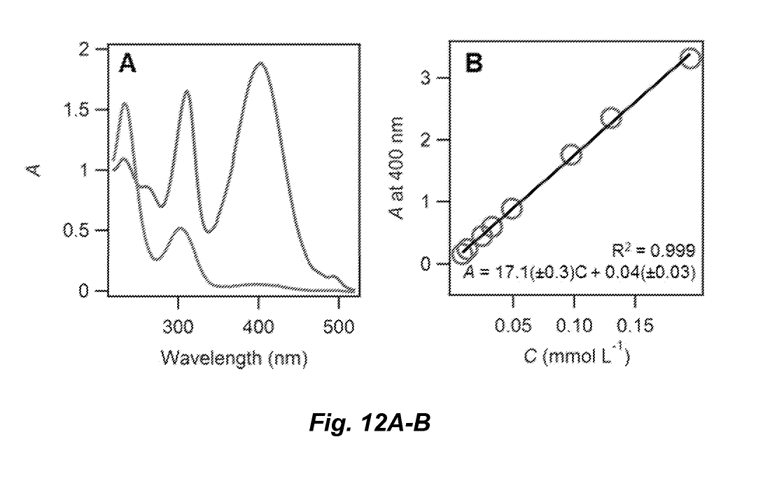

In addition to sorption, CNPs also exhibited the ability to catalyze the reduction of model pollutant p-nitrophenol (PNP) in the presence of a reducing agent, sodium borohydride (NaBH₄) (cf. FIG. 12A-B). United States Environmental Protection Agency, Clean Water Act Priority Pollutant List. 1982; p Code of Federal Regulations 40 CFR 423 Appendix A. As shown in FIG. 7A, CNP-catalyzed PNP reduction (cyan) followed pseudo first-order rate law mechanics when NaBH₄ was in excess: ln(C/Cₒ)=−kt, where C and Cₒ are residual and initial PNP concentrations and k is the reduction rate constant. Hong et al., Preparation and Microstructure Control of One-Dimension Core-Shell Heterostructure of Te/Bi, Te/Bi₂Te₃ by Microwave Assisted Chemical Synthesis. In Energy and Environment Materials, Tang, X. F.; Wu, Y.; Yao, Y.; Zhang, Z. Z., Eds. 2013, pp 153-160. Linear regression gave k=0.26(±0.01) min⁻¹ (R²=0.99). Adsorption made a negligible contribution to the PNP reduction as was evident from the flat line observed in the absence of borohydride (pink). CNPs' catalytic ability can be attributed to the Co nanoparticles in the supporting LDO disc. Sahiner et al., A Soft Hydrogel Reactor for Cobalt Nanoparticle Preparation and Use in the Reduction of Nitrophenols. Appl. Catal., B 2010, 101, 137-143.

To further improve CNPs' catalytic capability, 3-nm palladium (Pd) nanoparticles were decorated on CNPs at a density of 0.25(±0.01) g-Pd per g-CNP. The 1:4 Pd-to-C mass ratio was confirmed by measurements made with inductively coupled plasma optical emission spectroscopy after acid digestion. As shown in FIG. 7B, Pd nanoparticles were uniformly distributed on individual CNTs in CNPs. FIG. 7C shows a Pd nanoparticle oriented along the [110] zone axis under TEM. The Fast Fourier transform of the TEM image revealed distinctive electron diffractions from (002) and (111) planes, as shown in FIG. 7D, suggesting that Pd nanoparticles are singularly crystalline, which is represented by a truncated octahedral model, as shown in FIG. 7E. The presence of Pd nanoparticles has greatly enhanced the reduction of PNP, as shown in FIG. 7A (lower line, angling down), with a value of k=1.88(±0.08) min⁻¹ (R²=0.99).

After being normalized to the Pd mass, the value of k gave a rate constant of 608(±26) L min⁻¹ g⁻¹, which is comparable with literature values for Pd-catalyzed PNP reduction. Bhandari, R.; Knecht, M. R., Effects of the Material Structure on the Catalytic Activity of Peptide-Templated Pd Nanomaterials. ACS Catal. 2011, 1, 89-98; Harish et al., Synthesis of Conducting Polymer Supported Pd Nanoparticles in Aqueous Medium and Catalytic Activity Towards 4-Nitrophenol Reduction. Catal. Lett. 2009, 128, 197-202; Mei, Y.; Lu et al., Catalytic Activity of Palladium Nanoparticles Encapsulated in Spherical Polyelectrolyte Brushes and Core-Shell Microgels. Chem. Mater. 2007, 19, 1062-1069.

In summary, we have demonstrated that individual CNTs can be integrated into micrometer-sized colloidal particles without using heavy or bulky particulate support. The resulting carbon nanotube ponytails comprise CNTs grown on a nanometer-thin material disc having a negligible mass and volume. Compared to individual CNTs, CNPs can be more effectively separated from water using gravitational sedimentation, magnetic attraction, and membrane filtration, while having an improved ability to perform adsorption, disinfection, and catalytic degradation of contaminants in water. Organizing CNTs into hierarchical CNPs is a novel strategy to scale up nanomaterials for macroscopic engineering applications. CNPs can be used in treatment processes for water purification. They also can be deployed to combat accidental spills of chemical and biological contaminants.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Methods

Materials and methods used to synthesize, characterize, and evaluate materials in our experiments are described in this section. Gases were purchased from Airgas. All other chemicals were purchased from Sigma-Aldrich, unless stated otherwise. Information on the control of CNPs' morphology by varying synthesis parameters is provided.

### Example 1

### Synthesis of Carbon Nanotube Ponytails

Nitrate salts of aluminum, magnesium, and cobalt were mixed with urea in 100 mL deionized (DI) water (Millipore). The final concentrations of the precursor ingredients were 100 mmol L⁻¹ for urea and 50 mmol L⁻¹ for all metals: α% for Co, (67−α)% for Mg, and 33% for Al with a being varied from 5 to 33%. The solution was placed in a sealed autoclave reactor and heated to 100° C. After a period of time (typically 12 hours), layered double hydroxide (LDH) discs were produced. LDH discs were collected by centrifugation, washed with DI water, and calcined at 800° C. in air for 20 minutes. LDH discs were then placed inside a sealed quartz tubing and heated by a tube furnace to 800° C. under argon protection. Hydrogen was passed through the tubing at 50 sccm for 5 minutes to reduce LDH to LDO. Ethanol was then supplied by bubbling argon through a reservoir at 100 sccm for 15 minutes to grow CNT arrays on LDO discs.

### Example 2

### Synthesis of Unbounded Carbon Nanotubes

Unbounded CNTs used to compare with CNPs in gravitational settling were prepared using a powder catalyst consisting of cobalt, molybdenum, and magnesium. Wang et al., Removal of Oil Droplets from Contaminated Water Using Magnetic Carbon Nanotubes. Water Res. 2013, 47, 4198-4205. The growth of CNTs using CVD followed the same procedure as described above except that the powder catalyst was used instead of LDO discs. After 15 minutes of CVD growth, the powder catalyst was dissolved away by soaking CNTs in concentrated hydrochloric acid at 80° C. for 8 hours. The remaining CNTs were cleaned with DI water and freeze-dried (Labconco). The unbounded CNTs have similar morphologies and surface properties as the individual CNTs in CNPs, as described in more detail below.

### Example 3

### Preparation and Evaluation of Pd-Decorated CNPs

Nanoparticle decoration was achieved using a one-step protocol by mixing Pd(NO₃)₂ solution with CNPs. He, H. K.; Gao, C., A General Strategy for the Preparation of Carbon Nanotubes and Graphene Oxide Decorated with PdO Nanoparticles in Water. Molecules 2010, 15, 4679-4694. 10-mg CNPs were mixed with 20 mL DI water in a 50 mL flask under sonication. Twenty milliliters of Pd(NO₃)₂ solution (5 mM) were added to the flask drop by drop under magnetic stirring. The mixture was permitted to react for 30 minutes to form PdO nanoparticles on CNPs. PdO-CNPs were collected using an external magnetic field and washed repeatedly with DI water. The washed PdO-CNPs were re-dispersed in 40 mL water under sonication. PdO-CNPs were reduced to Pd-CNPs by mixing with sodium borohydride solution. The composition of PdO-CNPs was determined by dissolving the composite in concentrated nitric acid and measuring the Pd content using inductively coupled plasma optical emission spectroscopy (Perkin Elmer).

Material Characterization.

CNPs and other nanomaterials used in this study were also characterized using transmission electron microscope (FEI Titan), scanning electron microscope (FEI Magellan 400), powder X-ray diffractometer (Bruker D8 Advance Davinci), X-ray photoelectron spectroscopy (PHI 5000 VersaProbe), superconducting quantum interference device (Quantum Design MPMS SQUID), and surface area analyzer (Micromeritics ASAP2020). Sample preparation and analyses were performed following standard procedures.

### Example 4

### Gravitational and Magnetic Separation

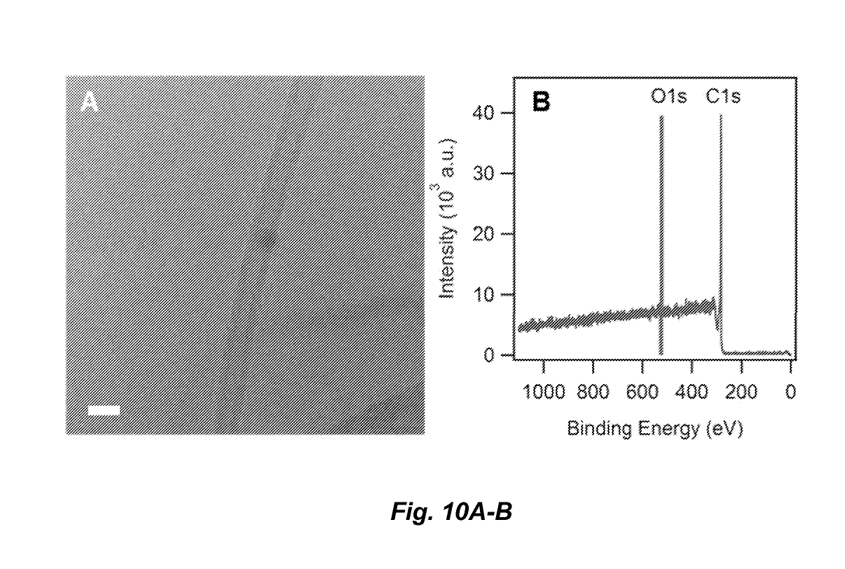

Gravitational sedimentation was performed in a 1 cm×1 cm quartz cuvette with a height of 2.5 cm of aqueous suspension. Light passed through a portion of the suspension from the top to 1.3 cm from the bottom. Carbon concentration in suspension was directly quantified by the absorbance of light at 500 nm (FIG. 10B). Magnetic separation was performed in a scintillation vial with a diameter of 2.8 cm using 15-mL of CNP suspension. The block magnet (K&J Magnetics BXOXOC) was placed to the side of the vial. The magnetic field inside the vial has an average strength of 4.2 kOe. To quantify the decrease of CNP concentration with time, 0.1 mL of suspension was taken periodically from the top of the suspension, diluted into 1 mL in a 2 mL quartz cuvette, and measured for light absorbance at 500 nm.

### Example 5

### Adsorption of Methylene Blue

Adsorption was quantified by measuring initial and residual MB concentrations, Cₒ and C, using light absorption at 664 nm after an incubation period t under shaking at room temperature. Bergmann, K.; Okonski, C. T., A Spectroscopic Study of Methylene Blue Monomer, Dimer, and Complexes with Montmorillonite. J. Phys. Chem. 1963, 67, 2169-2177.

In kinetic studies, 10 mg CNPs were added in 10 mL DI water in a glass vial. Solution pH was adjusted with concentrated HCl and NaOH solutions. MB stock solution (1000 ppm) was added to reach a total volume of 15 mL and mixed on a shaking table (300 rmp). 0.1 mL solution was periodically pipetted from the vial, filtered, and measured. After the adsorption experiment, pH was measured again, which was found to be within 0.3 pH unit from the initial pH. In equilibrium studies, 5 to 10 mg CNPs were added in 15 mL aqueous solution containing MB at a predetermined concentration. CNPs and MB were mixed under shaking for 4 hours. Solution pH was maintained at a preset value throughout the entire experimental duration using concentrated HCl and NaOH solutions. At the end of the experiment, CNPs were separated from treated water by a magnet and the MB concentrations were measured. Bergmann, K.; Okonski, C. T., A Spectroscopic Study of Methylene Blue Monomer, Dimer, and Complexes with Montmorillonite. J. Phys. Chem. 1963, 67, 2169-2177.

### Example 6

### Regeneration of Methylene Blue-Laden Carbon Nanotube Ponytails

CNPs (10 mg) were loaded with an equilibrium amount of MB in a 15 mL aqueous solution with a MB concentration of 120 mg L⁻¹ under vigorous shaking for 4 hours. CNPs were collected by magnetic separation. To evaluate the effectiveness of ethanol washes, CNPs were added to 15 mL ethanol under vigorous shaking To examine the desorption kinetics, 0.1-0.2 mL solution was taken by pipette periodically to measure the MB concentration in ethanol. The solution was dried in a scintillation vial by evaporating ethanol in a fume hood. The residual MB was re-dissolved in water for a concentration measurement. To examine the efficiency after the CNPs were regenerated by a 10-cycle ethanol wash, 6 mg of regenerated CNPs were mixed with 15 mL of MB aqueous solution (80 mg L⁻¹) for 4 hours. For thermal regeneration by microwave irradiation, CNPs were placed in a scintillation vial inside a kitchen microwave oven (R-209KK, Sharp Electronics Corp., Mahwan, N.J.; 800 W, 2.45 GHz) and the oven was turned on under full power for 3, 5, or 8 minutes.

### Example 7

### Removal of Escherichia coli

CNPs' ability to remove pathogenic bacteria was examined using E. coli DH5α. The bacterium was first cultivated in the LB liquid medium overnight. The culture was then washed in the phosphate buffered saline (PBS, Invitrogen). The wash was performed by adding 30 μL of the overnight culture into 30 mL of PBS. The washed bacteria were recollected using a centrifuge as cell pellets. The pellets were re-suspended in 30 mL of PBS to simulate contaminated water. CNPs were added to the simulated water in 4 mL vials. The mixture was first homogenized using a tissue grinder for 20 seconds and shaken for 1 hour. The mixture was then allowed to settle on a bench for 2 hours. Water was taken from the top layer for colony forming units (CFU) counting.

### Example 8

### Catalytic Reduction of p-Nitrophenol

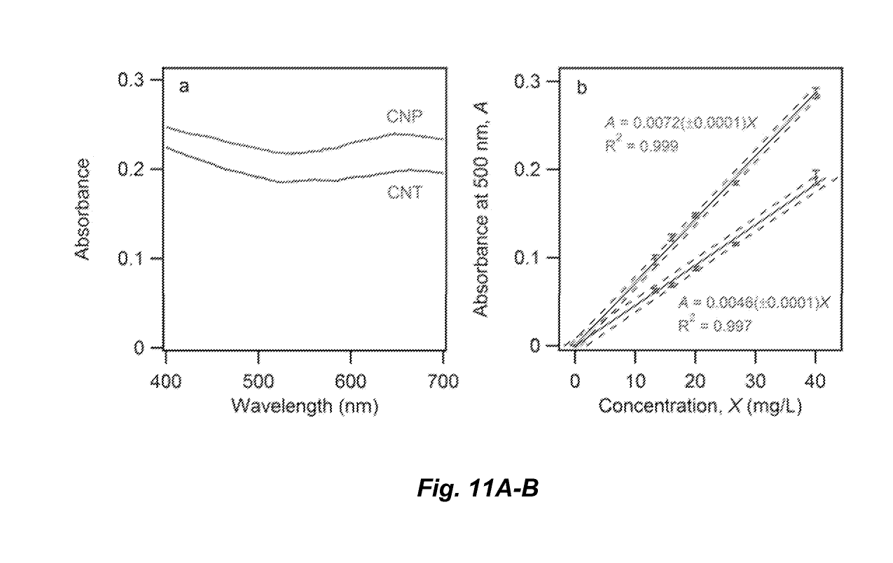

The reduction of PNP by sodium borohydride (SB) occurs rapidly in the presence of catalysts and can be readily followed using UV/vis spectrometry (FIG. 11A-B). Pradhan, N.; Pal, A.; Pal, T., Catalytic Reduction of Aromatic Nitro Compounds by Coinage Metal Nanoparticles. Langmuir 2001, 17, 1800-1802. 0.1 mL of well dispersed 0.25 g L⁻¹ CNPs solution or 0.31 g L⁻¹ Pd-decorated CNPs solution (equivalent amount of CNPs in both solutions), 1.9 mL NaBH₄ solution, and 0.02 mL 0.2-mM PNP were mixed in a standard quartz cuvette with a 1-cm path length. The concentration of PNP was monitored every 30 s for 5 min using light absorption at 400 nm. The solution was gently stirred with a glass rod in the catalytic process to avoid catalyst precipitation. An adsorption control experiment was conducted by replacing NaBH₄ solution with 0.0625 mol L⁻¹ NaOH solution, while keeping the other procedures identical.

### Example 9

### Structures of Layered Double Hydroxide and Layer Double Oxide Discs

The structures of LDH and LDO discs can be seen in FIGS. 8A and 8B.

### Example 10

### Control of Physical Dimensions of Carbon Nanotube Ponytails

The physical dimensions of CNPs can be tuned by varying parameters, such as synthesis time and cobalt doping. As illustrated in FIG. 9A, we have investigated the control of the radius of CNP cross section r, CNP's half-length l, CNT outer diameter d, and CNT wall number n. We measured r, l, d, and n from transmission electron micrographs of samples made under four different synthesis conditions. A set of measurements were used to create a histogram, which was fit to a Gaussian function to obtain estimates of the average value and standard deviation. An example of how the average and standard deviations of r were obtained is shown in FIG. 9B.

The radius of CNP cross section, r, was controlled by varying the time used to synthesize LDH through co-precipitation of Al, Mg, and Co hydroxides. An example is shown in FIG. 9C with 13% Co in the original reactive solution (i.e., α=13%). For syntheses that lasted less than 2 hours, we observed little LDH formation. With samples made between 2 and 4 hours, we observed a few measurable LDH discs and large amounts of small nuclei. We observed numerous LDH discs with further increase of synthesis time. The size length of the discs, which would be the radius of CNPs once CNTs were grown, increased monotonically with increasing synthesis time. Based on these observations, we concluded that the synthesis of LDH was dominated by the nucleation phase before 4 hours and then transitioned to the growth phase after 4 hours.

We modeled the growth of LDH by considering the rate-limiting step of co-precipitation, which is the hydrolysis of urea to carbonate:

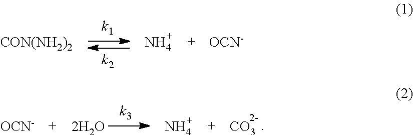

Warner, R. C., The Kinetics of the Hydrolysis of Urea and of Arginine. J. Biol. Chem. 1942, 142, 705-723.

The reactions are rate-limiting because carbonate is an intercalated anion that is required to fuse metal hydroxide sheets (cf. FIG. 9E). Accordingly, kinetics of Reactions S1 and S2 can be expressed as follows:

$$\begin{matrix}\frac{d [ {CO ( {NH}_{2} )}_{2} ]}{d t} = {k}_{1} [ {CO ( {NH}_{2} )}_{2} ] + {k}_{2} [ {NH}_{4}^{+} ] [ {OCN}^{-} ] & ( 3 ) \\ \frac{d [ {OCN}^{-} ]}{d t} = {k}_{1} [ {CO ( {NH}_{2} )}_{2} ] - {k}_{2} [ {NH}_{4}^{+} ] [ {OCN}^{-} ] - {k}_{3} [ {OCN}^{-} ] & ( 4 ) \\ \frac{d [ {CO}_{3}^{2 -} ]}{d t} = {k}_{3} [ {OCN}^{-} ] & ( 5 )\end{matrix}$$

We can neglect k₂[NH₄⁺][OCN⁻] in the above equations on the basis that the formation and growth of LDH discs are sinks of carbonate, which drive the overall reaction forward. As a result, k₁[CO(NH₂)₂]>>k₂[NH₄⁺][OCN⁻]. We further apply the pseudo steady-state condition for the reaction intermediate cyanate. Fogler, H. S., Elements of Chemical Reaction Engineering. Prentice Hall PTR: Upper Saddle River, N.J., 2000.

This gives the following equation:

$$\frac{d [ {OCN}^{-} ]}{d t} = {k}_{1} [ {CO ( {NH}_{2} )}_{2} ] - {k}_{3} [ {OCN}^{-} ] = 0. \qquad ( 6 )$$

After simplification, Equation 3 becomes:

$$\frac{d [ {CO ( {NH}_{2} )}_{2} ]}{d t} \approx - {k}_{1} [ {CO ( {NH}_{2} )}_{2} ] , \qquad ( 7 )$$

Integration of Equation 7 gives:

[CO(NH₂)₂]≈u₀(1−e^{-k}^{₁}^{t}) (8),

where u₀ is the initial urea concentration. Combining Equations 5, 6, and 8 gives:

$$\frac{d [ {CO}_{3}^{2 -} ]}{d t} = {k}_{3} [ {OCN}^{-} ] \approx {k}_{1} [ {CO ( {NH}_{2} )}_{2} ] = {k}_{1} {u}_{0} ( 1 - {e}^{- {k}_{1} t} ) . \qquad ( 9 )$$

Integration of Equation 9 from time 0 to t gives:

[CO₃²⁻]=u₀(k₁t+e^{-k}^{₁}^{t}−1) (10).

If we assume all carbonate produced by urea hydrolysis is taken up by LDH growth immediately after formation, we have the following equation:

[CO₃²⁻]V=3r²δηN (11),

where V is the reactor volume, r is the side length of LDH hexagons, δ is the thickness of LDH hexagons, η is the molar concentration of carbonate in LDH, and N is the number of LDH hexagons. We further assume that δ and N are determined at the early stage of LDH formation (i.e., t<10 hr), and thus, are constants at the later growth stage. Combining Equations 10 and 11 gives:

$$r = {A ( {k}_{1} t + {e}^{- {k}_{1} t} - 1 )}^{\frac{1}{2}} ; \\ A = {( \frac{{u}_{0} V}{3 δη N} )}^{\frac{1}{2}} . \qquad ( 12 )$$

The first term is a constant, while the second term reveals the dependence on t. Using the measured values of r at t≧10 hr, we estimate k₁=0.37(±0.24) hr⁻¹ from a least-square regression. This value is consistent with the first-order rate constant of 0.147 hr⁻¹ at circumneutral pH and 100° C. Warner, R. C., The Kinetics of the Hydrolysis of Urea and of Arginine. J. Biol. Chem. 1942, 142, 705-723.

We also obtained A=1.1(±0.4) μm. Using u₀=100 mmol L⁻¹, V=100 mL, δ=40(±16) nm, and η=1.53 mol L⁻¹ (for hydrotalcite), we estimate N=4.5(±3.7)×10⁷, which suggests that there are approximately 10 to 100 million LDH disks in 100 mL of reaction solution. The fitted model, together with its 68.3% percentile confidence intervals, is shown in FIG. 9C, which serves as a guideline to control the dimension of CNP cross section.

Different from synthesis time in co-precipitation that varies the size of the LDH discs, the variation of cobalt molar percentage in the reactive solution did not, however, change the size of the LDH discs (and consequently the size of the CNP cross section), as shown in FIG. 9D. This is consistent with the fact that Co, Mg, and Al are interchangeable in the LDH structure (cf. FIG. 8A). When the Co percentage was varied, the total amount of Co, Mg, and Al was kept constant.

For growing CNTs on LDO derived from LDH using chemical vapor deposition, as reaction time increased, most growth occurred in the first 15 min. After that, growth quickly reached a steady state. These results are shown in FIG. 9E with CNT growth expressed as the percentage of the steady-state mass. The observation that CNTs cease to grow after certain time in CVD can be attributed to blockage or poisoning of metal catalysts, which are Co nanoparticles in the synthesis system. Yasuda et al., Improved and Large Area Single-Walled Carbon Nanotube Forest Growth by Controlling the Gas Flow Direction. ACS Nano 2009, 3, 4164-4170; Reilly, P. T. A.; Whitten, W. B., The Role of Free Radical Condensates in the Production of Carbon Nanotubes During the Hydrocarbon Cvd Process. Carbon 2006, 44, 1653-1660.

Using 15 min as the growth time for CVD, we further investigated the change of CNP half-length l, CNT outer diameter d, and CNT wall number n as shown in FIGS. 9F, 9G, and 9H. Both d and n increased with increasing Co percentage, whereas l increased for α<20% and decreased for α>20%. The variations of l, d, and n with α can be rationalized by considering the increase in size of Co nanoparticles as α increases because Co nanoparticles were the catalysts from which CNTs were grown. As shown in FIG. 9I, the nominal diameter of Co nanoparticles, d_{Co}, increased with increasing α monotonically. Mass balance dictates that d_{Co} and α are related as follows:

d_{Co}=ρα^{1/3} (13),

where ρ is a constant determined by the dimensions of Co hollow spheres. We estimated that ρ=4.0 (±0.3) using a least-square regression (R²=0.97 with an intercept of 1.0 (±0.8) being essentially zero). The monotonic increases of d and n with increasing α can be attributed to the increase of d_{Co} with α because larger Co nanoparticles will catalyze the growth of CNTs with greater diameter and wall number. The positive correlation is also applicable to l and α at α<20%. However, at α>20%, increases of d and n dramatically increase the need of carbon mass. The expansion in the radial direction redirects carbon atoms that used to extend CNTs' length to increasing their diameters, which results in the decrease of l with increasing α for α>20%.

### Example 11

### Surface Hydrophobicity of Unbounded Carbon Nanotubes Determined Using X-Ray Photoelectron Spectroscopy

Surfaces of carbon nanotubes consisting of graphene sheets are intrinsically hydrophobic. When surfaces are functionalized with oxygen-containing groups, such as —COOH, —OH, and —O—, they become hydrophilic. With surfaces being hydrophobic or hydrophilic, CNPs and CNTs can have different affinities with water, which in turn affect their settling behavior in water. To exclude the possibility that surface wettability had affected settling of CNTs and CNPs in water, we performed X-ray photoelectron spectroscopy (XPS; PHI 5000 VersaProbe) measurements. In our measurements, we used the monochromatized Al Kα line (1486.6 eV) as incident X-ray. The standard deviation of peak position was determined to be approximately 0.05 eV.

As shown in FIGS. 2E and 10, both surfaces exhibited a strong C_{1S} peak at 284.6 eV, which were consist with a graphene surface having minimal functionalization. Okpalugo, T. I. T.; Papakonstantinou, P.; Murphy, H.; McLaughlin, J.; Brown, N. M. D., High Resolution Xps Characterization of Chemical Functionalised Mwcnts and Swcnts. Carbon 2005, 43, 153-161. The position for the O_{1S} peak had a reading within uncertainty of the baselines.

According to the width of the baseline, we determined that the O content of both samples was below 1.5%. According to one report, when surface O content was below 3%, carbon nanotubes were always neutral and hydrophobic from pH 5 to 9. Smith et al., Influence of Surface Oxides on the Colloidal Stability of Multi-Walled Carbon Nanotubes: A Structure-Property Relationship. Langmuir 2009, 25, 9767-9776. According to another report, when the O content was below 6%, carbon nanotubes were superhydrophobic. Aria, A. I. Control of Wettability of Carbon Nanotube Array by Reversible Dry Oxidation for Superhydrophobic Coating and Supercapacitor Applications. Ph.D., California Institute of Technology, Ann Arbor, 2013. Based on our measurements and these reports, we concluded both CNPs and CNTs used in our experiments had hydrophobic surfaces. Therefore, both samples had little affinity with water and their settling in water should be affected by surface hydrophobicity similarly. In other words, any difference in settling between CNPs and CNTs should be attributed to factors other than differences in surface properties. As stated above, we believe that differences in settling is due to the differences in size between CNPs and CNTs.

### Example 12

### Results of Least-Square Regressions in FIGS. 5 and 6 (Tables 1-6)

TABLE 1
Results of Linear Regressions in Figure 5A

| | | | | kₐ | | t for |
|---|---|---|---|---|---|---|
| | Cₒ | X | qₑ | (g mg⁻¹ | | q/qₑ = |
| pH | (mg L⁻¹) | (g L⁻¹) | (mg g⁻¹) | min⁻¹) | R² | 95% |
| 8 | 30 | 0.67 | 46(±1) | 0.017 | 0.999 | 24.3(±4.3) |
| | | | | (±0.003) | | |
| 6 | 30 | | | | | |
| 6 | 60 | | 84(±2) | 0.0028 | 0.996 | 80.8(±6.1) |
| | | | | (±0.0002) | | |
| 6 | 200 | | 148(±5) | 0.00036 | 0.995 | 357(±32) |
| | | | | (±0.00003) | | |

TABLE 2
Results of Linear Regression in FIG. 5B

| pH | Cₒ (mg L⁻¹) | X (g L⁻¹) | qₘₐₓ (mg g⁻¹) | K (L mg⁻¹) | R² |
|---|---|---|---|---|---|
| 4 − 10 | 60 − 200 | 0.5 − 1 | 150(±9) | 0.42(±0.42) | 0.98 |

TABLE 3
Results of Linear Regressions in Figure 5C

| | q_{o,n} | t_{o,n} | X | C_{e,n} | k_{d,n} | |
|---|---|---|---|---|---|---|
| n | (mg g⁻¹) | (min) | (g L⁻¹) | (mg L⁻¹) | (L mg⁻¹ min⁻¹) | R² |
| 1 | 98 | 0 | 0.67 | 29.8(±0.1) | 0.063(±0.002) | 1.000 |
| 2 | 53 | 120 | | 6.11(±0.02) | 0.052(±0.005) | 1.000 |
| 3 | 44 | 240 | | 3.49(±0.06) | 0.037(±0.003) | 0.998 |

TABLE 4
Results of Linear Regressions in FIG. 5D

| Cₒ (mg L⁻¹) | X (g L⁻¹) | dθ/dN (%) | R² |
|---|---|---|---|
| 80 | 0.4 | −1.9(±0.4) | 0.95 |

TABLE 5
Results of Linear Regressions in FIG. 5E

| Cycle | θ_{(8 min)} (%) | dθ/dt (% min⁻¹) | R² |
|---|---|---|---|
| 1 | 92 | 10(±2) | 0.97 |
| 2 | 48 | 6.9(±0.9) | 0.98 |
| 3 | 4 | 0.6(±0.2) | 0.87 |

TABLE 6
Results of Linear Regression in FIG. 6B

| pH | Nₒ (CFUs mL⁻¹) | X(g L⁻¹) | qₘₐₓ (CFUs g⁻¹) | K (L g⁻¹) | R² |
|---|---|---|---|---|---|
| 7 | 1.3 × 10⁵ | 0 − 0.2 | 2.3(±0.2) × 10⁹ | 0.2 (±0.2) | 0.97 |

### Example 13

### Quantification of Carbon Nanotubes and Carbon Nanotube Ponytails in Aqueous Suspensions Using UV/Vis Spectrometry

Carbon nanotubes and carbon nanotube ponytails are good absorbents of visible light, as indicated by their black color and shown by the intensive absorption of light from 400 to 700 nm using a Cary 100 UV/vis spectrophotometer, as shown in FIG. 11A. The light-absorbing property was utilized to quantify concentrations of CNTs and CNPs suspended in water by sonication (5 min). We selected 500 nm as the wavelength in the measurement, although light with other wavelengths between 400 and 700 nm should also work.

To make a calibration curve that can relate light absorbance at 500 nm to the concentration of CNTs or CNPs suspended in water, we mixed different amounts of CNTs or CNPs with 50 mL of DI water under sonication for 5 minutes. To obtain the accurate mass of CNTs or CNPs, the samples were freeze-dried before weighing. As shown in FIG. 11B, absorbance and concentration have linear relationships for both CNTs and CNPs. We modeled the linear relationship using Beer's law:

A=εXL (14),

where A=log(I/I₀) is absorbance, I₀ is the intensity of the incident light, I is the intensity of the transmitted light, ε is the extinction coefficient, X is the concentration, and L is the length of the light path (L=1 cm in our experiments). According to the slopes of the absorbance-concentration linear relationships, we estimate the specific extinction coefficients of water-dispersed CNTs and CNPs to be ε_{CNT}=4.6(±0.1) cm² mg⁻¹ and ε_{CNP}=7.2(±0.1) cm² mg⁻¹, respectively.

Both estimates are consistent with the values of extinction coefficients for well-dispersed CNTs. Bahr et al., Dissolution of Small Diameter Single-Wall Carbon Nanotubes in Organic Solvents? Chem. Commun. 2001, 193-194; Roldo et al., N-Octyl-O-Sulfate Chitosan Stabilises Single Wall Carbon Nanotubes in Aqueous Media and Bestows Biocompatibility. Nanoscale 2009, 1, 366-373; Liu et al., Functionalization of Single-Walled Carbon Nanotubes with Well-Defined Polymers by Radical Coupling. Macromolecules 2005, 38, 1172-1179; Zhou et al., Absorptivity of Functionalized Single-Walled Carbon Nanotubes in Solution. J. Phys. Chem. B 2003, 107, 13588-13592.

Average settling velocity v can be obtained from the change of X with t according to the following equation:

$$\frac{V d X}{d t} = α v X , \qquad ( 15 )$$

where V is the volume of the suspension and a is cross-section area of the settling vial. This equation relates the flux of CNTs or CNPs settled out of the suspension with the flux at the bottom of the suspension. Integration of Equation 15 gives:

$$ln \frac{X}{{X}_{o}} = - \frac{α}{V} vt = - \frac{v}{h} t , \qquad ( 16 ) ,$$

where h is the height of the suspension.

### Example 14

### Quantification of p-Nitrophenol Reduction Using UV/VIS Spectrometry

The reduction of PNP by sodium borohydride (NaBH₄; SB) to p-aminophenol (PAP) is a well-studied reaction:

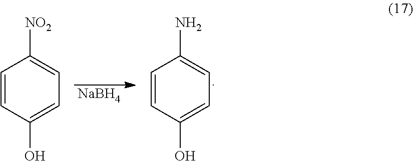

### Pradhan et al., Langmuir 2001, 17, 1800-1802.

The hydrogenation reaction is greatly promoted by the presence of catalysts, such as Pd nanoparticles (PdNPs), whose surface facilitates the generation of hydrogen. The procession of this reaction can be readily detected by the naked eye as the yellow color of PNP fades away with time in the presence of excess SB. As shown in FIG. 12A, the absorption spectrum of PNP in NaBH₄ peaked at 400 nm, due to the formation of p-nitrophenolate from dissociation (pKₐ=7.2). Liu et al., Chem.-Eur. J. 2006, 12, 2131-2138. In comparison, PAP absorbs minimal light from 325 to 600 nm. Dotzauer et al., Nanoparticle-Containing Membranes for the Catalytic Reduction of Nitroaromatic Compounds. Langmuir 2009, 25, 1865-1871; Ballarin et al., Gold Nanoparticle-Containing Membranes from in Situ Reduction of a Gold(III)-Aminoethylimidazolium Aurate Salt. J. Phys. Chem. C 2010, 114, 9693-9701. This indicates that the absorbance at 400 nm can be used to quantify the PNP concentration according to Beer's law (cf. Equation 14), as shown by the calibration curve in FIG. 11B. Measuring PNP concentration periodically as time passes provides measurements of the kinetics of Reaction 17.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. Carbon nanotube ponytail (CNP) particles comprising a carbon nanotube (CNT) array integrated onto a support and having:
(a) a mass ratio of CNT array:support of greater than about 90%;
(b) a volume ratio of CNT array:support of greater than about 90%; and
(c) a CNP particle length of about 1-500 μm.

2. The CNP particles of claim 1 having:
(a) a mass ratio of CNT array:support of greater than about 95%;
(b) a volume ratio of CNT array:support of greater than about 95%; and
(c) a CNP particle length of about 1-200 μm.

3. The CNP particles of claim 1 having lengths of about 1-200 μm and diameters of about 0.1-10 μm.

4. The CNP particles of claim 3 having lengths of about 50-200 μm and diameters of about 1-5 μm.

5. The CNP particles of claim 1 wherein the support has a thickness of about 10-100 nm and a diameter of about 0.1-5 μm.

6. The CNP particles of claim 5 wherein the support has a thickness of about 20-80 nm and a diameter of about 1-5 μm.

7. The CNP particles of claim 1 wherein each CNP particle comprises two arrays of CNT particles that are entangled and integrated onto the support.

8. The CNP particles of claim 1 having pore sizes of about 4.5-100 nm and specific surface areas (SSAs) of about 200-600 m² g⁻¹.

9. The CNP particles of claim 1 wherein the support comprises layered double oxide (LDO).

10. The CNP particles of claim 9 wherein the LDO support comprises cobalt or iron nanoparticles.

11. The CNP particles of claim 10 wherein the LDO support further comprises magnesium and aluminum.

12. The CNP particles of claim 11 wherein the LDO support further comprise precious metal or noble metal nanoparticles.

13. The CNP particles of claim 1 functionalized with oleophillic moieties.

14. A water or waste treatment system comprising the CNP particles of claim 1.

15. A method of treating a contaminated liquid comprising exposing the contaminated liquid to carbon nanotube ponytail (CNP) particles and separating the treated liquid from the CNP particles, wherein the CNP particles comprise a carbon nanotube (CNT) array integrated onto a support and have:
(a) a mass ratio of CNT array:support of greater than about 90%;
(b) a volume ratio of CNT array:support of greater than about 90%; and
(c) a CNP particle length of about 1-500 μm.

16. The method of claim 15 wherein the separation step is accomplished by (i) gravitational sedimentation, (ii) magnetic attraction, (iii) membrane filtration, or (iv) a combination thereof.

17. The method of claim 15 wherein the treatment comprises adsorbing the contaminants onto the CNP particles or catalyzing the contaminants to a less noxious state.

18. The method of claim 15, wherein after separation, the CNP particles are regenerated for re-use via a solvent wash or thermal exposure.

19. A method of making carbon nanotube ponytail (CNP) particles comprising:
(i) co-precipitating aluminum, magnesium, and cobalt or iron cations with hydroxide and carbonate anions to form a layered double hydroxide (LDH) support;
(ii) reducing the LDH support to a layered double oxide (LDO) support; and
(iii) growing an entangled carbon nanotube (CNT) array that is integrated onto the LDO support to form the CNP particles;
wherein the CNP particles have:
(a) a mass ratio of CNT array:support of greater than about 90%;
(b) a volume ratio of CNT array:support of greater than about 90%; and
(c) a CNP particle length of about 1-500 μm.

20. The method of claim 19 wherein
(i) the LDH discs are prepared by mixing and heating a solution of nitrate salts of aluminum, magnesium, and cobalt or iron with urea in deionized water;
(ii) the LDO discs are prepared by dehydrating and decarbonating the LDH discs; and
(iii) the CNT particles are prepared by using ethanol as a carbon source.
